(19) United States
(12) Patent Application Publication
(10) Pub. No.: US 2025/0205442 A1
(43) Pub. Date: Jun. 26, 2025

(54) CUSHIONS FOR PATIENT INTERFACES

(71) Applicant: ResMed Pty Ltd, Bella Vista, New South Wales (AU)

(72) Inventors: Kelvin Dale ALPHONSO, Sydney (AU); Alexander Graham BENNETT, Sydney (AU); Callum Tyler DE VRIES, Sydney (AU); David John FRANK, Sydney (AU); Kam Man LAW, Sydney (AU); Nigel QUARMBY, Sydney (AU); Alexandra Elizabeth TILSON, Sydney (AU); Michal Konrad TREFLER, Sydney (AU); Andrew James BATE, Sydney (AU); Jeremy David MCMANUS, Sydney (AU)

(21) Appl. No.: 18/848,464

(22) PCT Filed: Nov. 29, 2023
(86) PCT No.: PCT/AU2023/051224

§ 371 Date: Sep. 18, 2024

(30) Foreign Application Priority Data
Nov. 29, 2022 (AU) 2022903613
Dec. 20, 2022 (AU) 2022903915
Nov. 6, 2023 (AU) 2023903550

(51) Int. Cl. A61M 16/06

(52) U.S. Cl. CPC A61M 16/0622; A61M 2205/0216

(57) ABSTRACT

Cushion modules and cushion module components are provided for use with patient interfaces in respiratory therapy systems. In examples the seal-forming structures of the cushion modules are provided with a reduced thickness region which may provide improved patient comfort and/or improved manufacturing techniques, particularly when used in relation to patient interfaces combining two or more different materials. In some examples the cushion comprises a lap joint between two different materials.

### 1 CROSS-REFERENCE TO RELATED APPLICATIONS

This application claims the benefit of, and priority to, Australian Provisional Patent Application No. 2023903550, filed on 6 Nov. 2023, Australian Provisional Patent Application No. 2022903613, filed on 29 Nov. 2022, and Australian Provisional Patent Application No. 2022903915, filed on 20 Dec. 2022, each of which are hereby incorporated by reference in their entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use. The present technology also relates to patient interfaces and cushion modules therefore.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth; if they require a patient to insert a portion of a mask structure in their mouth to create and maintain a seal via their lips; and/or if they are impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal-forming structure, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO 2004/073778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063328 and WO 2006/130903 (describing amongst other things aspects of the ResMed Limited MIRAGE LIBERTY™ full-face mask); International Patent Application WO 2009/052560 (describing amongst other things aspects of the ResMed Limited SWIFT™ FX nasal pillows).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 2.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming structure of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has an opening therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a cushion module for a patient interface.

Another form of the present technology comprises a cushion module for a patient interface comprising a reduced thickness region in the seal-forming structure of the patient interface.

Another form of the present technology comprises a patient interface for delivering a flow of breathable gas to the airways of a patient comprising: a connection port configured to receive the flow of breathable gas, a cushion module which includes a plenum chamber configured to receive the flow of breathable gas, and a seal-forming structure configured to form a seal with the airways or around the airways of the patient, wherein the seal-forming structure comprises a patient contacting portion configured to engage with the patient's face to provide the seal, and a support portion attached to a non-patient contacting side of the patient contacting portion, the support portion being configured to support the patient contacting portion, and wherein the support portion comprises a reduced thickness region which extends substantially parallel to at least a portion of an outer perimeter of the patient contacting portion.

Another form of the present technology comprises a patient interface for delivering a flow of breathable gas to the airways of a patient, comprising: a plenum chamber pressurisable to a therapeutic pressure of at least 6 cmH2O above ambient air pressure, the plenum chamber comprising at least one plenum chamber inlet port being sized and structured to receive a flow of breathable gas at the therapeutic pressure for breathing by a patient, and a seal-forming structure constructed and arranged to form a seal with a region of the patient's face surrounding an entrance to the patient's airways, the seal-forming structure constructed and arranged to maintain said therapeutic pressure in the plenum chamber throughout the patient's respiratory cycle in use, wherein the seal-forming structure comprises a first region having a first thickness, at least one opening provided in the first region such that the flow of breathable gas is delivered to one or more of the patient's airways through the opening in use, and a second region which is contiguous with at least a portion of the first region, the second region having a thickness which is less than the first thickness.

Another form of the present technology comprises a cushion module for a patient interface, the cushion module comprising: a plenum chamber pressurisable to a therapeutic pressure of at least 6 cmH2O above ambient air pressure, the plenum chamber comprising at least one plenum chamber inlet port being sized and structured to receive a flow of breathable gas at the therapeutic pressure for breathing by a patient, and a seal-forming structure at least partially defining the plenum chamber and being constructed and arranged to form a seal with a region of the patient's face surrounding an entrance to the patient's airways, the seal-forming structure constructed and arranged to maintain said therapeutic pressure in the plenum chamber throughout the patient's respiratory cycle in use. In examples the seal-forming structure may comprise a first portion made from a first material, at least one opening provided in the first portion through which the flow of breathable gas is provided to one or more of the patient's airways in use, and a second portion comprising a second material, wherein the second portion is joined to the first portion, the second portion having at least one groove or channel therein, wherein the groove or channel lies along a path that is generally parallel to at least a portion of the opening.

Another form of the present technology comprises a cushion module for a patient interface comprising: a plenum chamber pressurisable to a therapeutic pressure of at least 6 cmH2O above ambient air pressure, the plenum chamber comprising at least one plenum chamber inlet port being sized and structured to receive a flow of breathable gas at the therapeutic pressure for breathing by a patient, and a seal-forming structure at least partially defining the plenum chamber and being constructed and arranged to form a seal with a region of the patient's face surrounding an entrance to the patient's airways, the seal-forming structure constructed and arranged to maintain said therapeutic pressure in the plenum chamber throughout the patient's respiratory cycle in use. In examples, the seal-forming structure may comprise a patient contacting portion comprising a first material, the patient contacting portion comprising a first side configured to engage the patient's face to form the seal, and a second side which is opposite to the first side, and an opening through which the flow of breathable gas is provided to the patient's airways in use. In examples the seal-forming structure further comprises a support portion attached to the second side of the patient contacting portion, the support portion comprising a second material which is different to the first material. In examples the support portion is configured to support at least part of the patient contacting portion, and the support portion comprises at least one reduced thickness region which has a thickness which is less than a thickness of an adjacent region of the support portion, the adjacent region being closer to the opening than the reduced thickness region, and the reduced thickness region lies on a path which extends in a direction which is generally parallel to the opening.

In certain forms, the reduced thickness region may be substantially narrow (in examples, less than 5 mm wide, such as approximately 1 mm to approximately 2 mm wide) and elongate (in examples, at least 20 mm long). In examples, the ratio of length to width of the reduced thickness region may be at least 4:1. The reduced thickness region may extend through one or more of a side-of-nose region; a nasal bridge region; an upper lip region; cheek regions; a lower lip regions; chin regions and/or forehead regions of the patient interface. For example, in one form the reduced thickness region may be provided in the side of nose and nasal bridge regions of the patient interface.

In certain forms, the patient interface may comprise a plurality of reduced thickness regions. While in other forms the patient interface may comprise a reduced thickness region which forms a closed curve or continuous loop around an opening in the cushion module.

Another aspect of one form of the present technology is a patient interface comprising:
a plenum chamber pressurisable to a therapeutic pressure of at least 6 cmH₂O above ambient air pressure, the plenum chamber comprising at least one plenum chamber inlet port being sized and structured to receive a flow of air at the therapeutic pressure for breathing by a patient;
a seal-forming structure constructed and arranged to form a seal with a region of the patient's face surrounding an entrance to the patient's airways, the seal-forming structure having at least one opening therein such that the flow of air at the therapeutic pressure is delivered to at least an entrance to the patient's nares, the seal-forming structure constructed and arranged to maintain said therapeutic pressure in the plenum chamber throughout the patient's respiratory cycle in use;
a vent to allow a flow of gases exhaled by the patient from an interior of the plenum chamber to ambient, said vent being sized and shaped to maintain the therapeutic pressure in the plenum chamber in use;
wherein the seal-forming structure comprises:
   a first layer on a posterior side of the seal-forming structure, the first layer configured to seal in use around the entrance to the patient's airways including against an at least partially inferior-facing portion of the patient's pronasale, against the nasal alae and against the lip superior of the patient's face in use;
   a second layer connected to and supporting the first layer, the second layer comprising an overlapping portion overlapping with the first layer to form a lap joint with the first layer around a periphery of the first layer, the overlapping portion extending inwardly with respect to the periphery of the first layer from a non-overlapping portion of the second layer;
   wherein the overlapping portion comprises a width which varies along the overlapping portion around the periphery of the first layer, the width being defined by the amount of inward extension of the overlapping portion from the non-overlapping portion of the second layer.

In further examples:
the overlapping portion comprises a pair of lateral nasal portions, the width of the overlapping portion being larger in the lateral nasal portions than in one or more other portions of the overlapping portion;
the width of the overlapping portion is larger in the lateral nasal portions than in a superior portion of the overlapping portion;
the overlapping portion is at least twice as wide in the lateral nasal portions than in the superior portion;
the width of the overlapping portion is larger in the lateral nasal portions than in an inferior portion of the overlapping portion;
the overlapping portion is at least five times as wide in the lateral nasal portions than in the inferior portion;
the overlapping portion is at least ten times as wide in the lateral nasal portions than in the inferior portion;
the width of the overlapping portion is larger in the superior portion than in the inferior portion of the overlapping portion;
the overlapping portion comprises a pair of mid-lateral inferior portions located on respective lateral sides of the inferior portion of the overlapping portion and located medially of the lateral nasal portions of the overlapping portion, the width of the overlapping portion being larger in the lateral nasal portions than the mid-lateral inferior portion;
the overlapping portion is at least 1.5 times as wide in the lateral nasal portions than in the mid-lateral inferior portions;
the width of the overlapping portion is larger in the mid-lateral inferior portions than in the inferior portion of the overlapping portion;
the seal-forming structure comprises a nasal portion configured to provide the flow of air to the entrance to the patient's nares in use, and further comprise an oral portion configured to provide the flow of air to the patient's mouth in use;
the overlapping portion comprises a pair of lateral lip superior portions provided at respective lateral sides of the patient's lip superior in use;
the width of the overlapping portion is larger in the lateral lip superior portions than in the lateral nasal portions;
the overlapping portion is at least 1.5 times as wide in the lateral lip superior portions than in the lateral nasal portions;
the lateral lip superior portions extend medially of the periphery of the first layer from a non-overlapping portion of the second layer;
each lateral lip superior portion comprises a medial end positioned proximate and inferior to a respective one of the alar crest points on the patient's face;
the width of the overlapping portion tapers between the lateral lip superior portions and the lateral nasal portions;
the overlapping portion comprises a pair of nasolabial portions each located laterally of a respective one of the lateral lip superior portions, the width of the overlapping portion being larger in the lateral lip superior portions than in the nasolabial portions;
the width of the overlapping portion is larger in the lateral nasal portions than in the nasolabial portions;
the overlapping portion comprises a pair of cheek portions, the width of the overlapping portion being larger in the lateral lip superior portions than in the cheek portions;
the overlapping portion comprises a lip inferior portion, the width of the overlapping portion being larger in the lateral lip superior portions than in the lip inferior portion;
the widths of the lateral nasal portions taper towards the superior portion of the overlapping portion;
the overlapping portion of the second layer comprises an exterior surface to which the first layer is connected, the exterior surface being offset from and shy of an exterior surface of the non-overlapping portion;
a patient-contacting surface of the first layer is flush with the exterior surface of the non-overlapping portion of the second layer;
the overlapping portion of the second layer of the seal-forming structure has a lesser thickness than the non-overlapping portion of the second layer of the seal-forming structure in one or more locations;
the first layer is formed from a textile material;
the second layer is formed from an elastomeric material;
the patient interface comprises a cushion module, the cushion module comprising a chassis portion, the seal-forming structure being attached to the chassis portion, the chassis portion and seal-forming structure together defining the plenum chamber;
the chassis portion comprises a pair of laterally projecting connection portions each defining a respective plenum chamber inlet port and each being configured to connect to a respective one of a pair of gas delivery tubes; and/or
the patient interface comprises the gas delivery tubes, the gas delivery tubes forming part of a positioning and stabilising structure configured to provide a force to hold the seal-forming structure in a therapeutically effective position on the patient's head, each gas delivery tube being configured to convey the flow of air from a location atop the patient's head to the plenum chamber in use.

Another aspect of one form of the present technology is a patient interface comprising:
a plenum chamber pressurisable to a therapeutic pressure of at least 6 cmH₂O above ambient air pressure, the plenum chamber comprising at least one plenum chamber inlet port being sized and structured to receive a flow of air at the therapeutic pressure for breathing by a patient;
a seal-forming structure constructed and arranged to form a seal with a region of the patient's face surrounding an entrance to the patient's airways, the seal-forming structure having at least one opening therein such that the flow of air at the therapeutic pressure is delivered to at least an entrance to the patient's nares, the seal-forming structure constructed and arranged to maintain said therapeutic pressure in the plenum chamber throughout the patient's respiratory cycle in use;
a vent to allow a flow of gases exhaled by the patient from an interior of the plenum chamber to ambient, said vent being sized and shaped to maintain the therapeutic pressure in the plenum chamber in use;
wherein the seal-forming structure comprises:
   a central portion configured to seal in use against an at least partially inferior-facing portion of the patient's pronasale, against the nasal alae and against the lip superior of the patient's face in use;
   a central anterior portion located proximate and inferior to the patient's pronasale in use in an anterior-facing wall of the seal-forming structure;
   a pair of mid-lateral anterior portions in the anterior-facing wall positioned on respective lateral sides of the central anterior portion, each of the mid-lateral anterior portions comprising a superior portion and an inferior portion, the inferior portion having a lesser stiffness than the superior portion.

In examples:
the inferior portion of each mid-lateral anterior portion has a lesser thickness than the superior portions of the mid-lateral anterior portions;
the inferior portion of each mid-lateral anterior portion has a greater stiffness than the central anterior portion;
the inferior portion of each mid-lateral anterior portion has a greater thickness than the central anterior portion;
the central portion is formed from a textile material;
the patient interface comprises a first layer provided to a posterior side of the seal-forming structure, the first layer forming the central portion of the seal-forming structure, and a second layer connected to and supporting the first layer around a periphery of the first layer, the second layer forming the anterior-facing wall of the seal-forming structure;
the first layer is formed from a textile material;
the second layer is formed from an elastomeric material;
the second layer of the seal-forming structure comprises an overlapping portion overlapping with the first layer to form a lap joint with the first layer around a periphery of the first layer, the overlapping portion extending inwardly with respect to the periphery of the first layer from a non-overlapping portion of the second layer;
the overlapping portion of the second layer comprises a reduced thickness region extending along at least a portion of the overlapping portion;
the reduced thickness region extends along the overlapping portion of the second layer in at least lateral nasal portions and superior portions of the overlapping portion;
the patient interface comprises a cushion module, the cushion module comprising a chassis portion, the seal-forming structure being attached to the chassis portion, the chassis portion and seal-forming structure together defining the plenum chamber;
the seal-forming structure comprises a pair of lateral posterior regions provided on respective lateral posterior sides of the seal-forming structure, each lateral posterior region extending posteriorly from the chassis portion and curving medially into contact with the patient's face in use;
each lateral posterior region curves medially to form a respective one of a pair of posterior corners of the seal-forming structure, each posterior corner being configured to engage the patient's face proximate a respective one of the patient's nasolabial sulci in use;
the seal-forming structure comprises a lip superior portion comprising a posterior portion configured to seal in use against the patient's lip superior and an anterior portion adjacent the chassis portion having a stiffness greater than a stiffness of the posterior portion of the lip superior portion;
the chassis portion comprises a pair of laterally projecting connection portions each defining a respective plenum chamber inlet port and each being configured to connect to a respective one of a pair of gas delivery tubes; and/or
the patient interface comprises the gas delivery tubes, the gas delivery tubes forming part of a positioning and stabilising structure configured to provide a force to hold the seal-forming structure in a therapeutically effective position on the patient's head, each gas delivery tube being configured to convey the flow of air from a location atop the patient's head to the plenum chamber in use.

Another aspect of one form of the present technology is a patient interface for delivering a flow of breathable gas to the airways of a patient, the patient interface comprising:
a plenum chamber pressurisable to a therapeutic pressure of at least 6 cmH2O above ambient air pressure, the plenum chamber comprising at least one plenum chamber inlet port being sized and structured to receive a flow of air at the therapeutic pressure for breathing by a patient;
   a seal-forming structure constructed and arranged to form a seal with a region of the patient's face surrounding an entrance to the patient's airways, the seal-forming structure having at least one opening therein such that the flow of air at the therapeutic pressure is delivered to at least an entrance to the patient's nares, the seal-forming structure constructed and arranged to maintain said therapeutic pressure in the plenum chamber throughout the patient's respiratory cycle in use;
wherein the seal-forming structure comprises a first layer configured to engage and seal with the face of the patient in use, and a second layer configured to provide support to at least a portion of the first layer in use,
wherein the first layer includes a first region which is joined to the second layer, and a second region which is not joined to the second layer,
wherein the second layer comprises a first thickness and a second thickness, wherein the first thickness is adjacent to the second region of the first layer, and the second thickness is both adjacent to the first thickness, and further from the second region than the first thickness, and
wherein first thickness is greater than the second thickness.

In some examples, the first region may be joined to the second layer with a lap joint.

In some examples, the second layer may comprise a third thickness which is adjacent to the second thickness, and further from the second region than the second thickness. In some examples, the first thickness may be greater than the third thickness.

In some examples, the first and/or second thicknesses may be provided as a channel having an elongate structure which extends in a circumferential direction around at least a portion of the seal-forming structure. For example, the elongate structure may form a continuous loop around the at least one opening in the seal-forming structure.

In some examples, the first and second thicknesses may be provided in one or more of a: side-of-nose region; a nasal bridge region; an upper lip region; cheek region; a lower lip region; a chin region; an alar region; and/or a pronasale region of the seal-forming structure.

In some examples, the first layer may comprise a textile and the second layer comprises an elastomeric material.

In some examples, the second layer may comprise a material which has a Young's modulus of 0.4 GPa or lower. For example the material may be a foam, silicone or rubber.

Another aspect of one form of the present technology is a patient interface for delivering a flow of breathable gas to the airways of a patient, the patient interface comprising:
a plenum chamber pressurisable to a therapeutic pressure of at least 6 cmH2O above ambient air pressure, the plenum chamber comprising at least one plenum chamber inlet port being sized and structured to receive a flow of air at the therapeutic pressure for breathing by a patient;
a seal-forming structure constructed and arranged to form a seal with a region of the patient's face surrounding an entrance to the patient's airways, the seal-forming structure constructed and arranged to maintain said therapeutic pressure in the plenum chamber throughout the patient's respiratory cycle in use;
wherein the seal-forming structure comprises an edge which defines an opening such that the flow of air at the therapeutic pressure is delivered from the plenum chamber through the opening to the patient's airways,
wherein the seal-forming structure comprises a patient contacting portion configured to engage with the patient's face to provide the seal, and a support portion attached to a non-patient contacting side of the patient contacting portion, the support portion being configured to support the patient contacting portion,
wherein the support portion comprises a first region and a second region, the first region being thinner than the second region, and further wherein the second region is closer to the edge of the seal-forming structure than the first region.

In some examples the first region has a width and a length, the length being measured in a circumferential direction around the seal-forming structure, and the width being measured across a surface of the seal-forming structure in a direction substantially perpendicular to the circumferential direction, wherein the length substantially greater than the width.

In some examples, the patient interface further comprises a third region, the third region being thicker than the first region and being located further from the edge of the seal-forming structure than the first region. The third region may extend in a circumferential direction around the seal-forming structure.

In some examples, the first region is provided in one or more of a: side-of-nose region; a nasal bridge region; an upper lip region; cheek region; a lower lip region; a chin region and/or a pronasale region of the seal-forming structure. For example, the first region may be provided in the side of nose and nasal bridge regions of the patient interface.

In some examples, the support portion comprises a plurality of first regions including said first region and a plurality of second regions including said second region, each of the first regions having a thickness which is less than a corresponding one of the second regions. The plurality of first regions are provided on opposite sides of the seal-forming structure in a substantially symmetrical arrangement.

In some examples, the first region forms a continuous loop around the opening in the seal-forming structure.

In some examples, a region of transition from the first region to the second region has a substantially curved profile when viewed in a cross-sectional plane extending substantially perpendicular to a longitudinal direction of the first region.

In some examples, the first region is provided as a channel in the non-patient contacting side of the patient contacting portion.

In some examples, the thickness of the first region varies around the opening in the seal-forming structure.

In some examples, the patient contacting portion comprises a textile.

Another aspect of one form of the present technology is a patient interface for delivering a flow of breathable gas to the oral and nasal airways of a patient, the patient interface comprising:
a plenum chamber pressurisable to a therapeutic pressure of at least 6 cmH2O above ambient air pressure, the plenum chamber comprising at least one plenum chamber inlet port being sized and structured to receive a flow of air at the therapeutic pressure for breathing by a patient;
   a seal-forming structure constructed and arranged to form a seal with a region of the patient's face surrounding an entrance to the patient's airways, the seal-forming structure constructed and arranged to maintain said therapeutic pressure in the plenum chamber throughout the patient's respiratory cycle in use;
wherein the seal-forming structure comprising a textile patient contacting layer, wherein the textile patient contacting layer comprises:
a nasal region which surrounds one or more nasal openings configured to deliver a flow of breathable gas to the nares of a patient in use, and an oral region which surrounds one or more oral openings configured to deliver a flow of breathable gas to the mouth of a patient in use,
wherein the textile patient contacting layer in the nasal region has a first width which is measured from the nasal opening(s) radially outwardly to an outer edge of the textile patient contacting layer, and wherein the textile patient contacting layer in the oral region has a second width which is measured from the oral opening(s) radially outwardly to an outer edge of the textile patient contacting layer in a chin region or a side of mouth region,
wherein the textile patient contacting layer has a third width which is measured from the oral opening to an outer edge of the textile patient contacting layer in a region where the upper lip meets a side of mouth region of the first layer,
wherein the third width is less than the first width and second width, and wherein the third width is configured to allow the textile patient contacting layer to curve from the oral region into the nasal region, and also assume a concave shape in the nasal region.

In some examples, the third width may be less than or equal to 50% of the second width.

In some examples the first, second and third widths may be measured with the textile patient contacting layer in a planar configuration.

In some examples the seal-forming structure may comprise a second layer configured to support at least a portion of the textile patient contacting layer.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure and connecting the positioning and stabilizing structure to either a first cushion module or a second cushion module.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

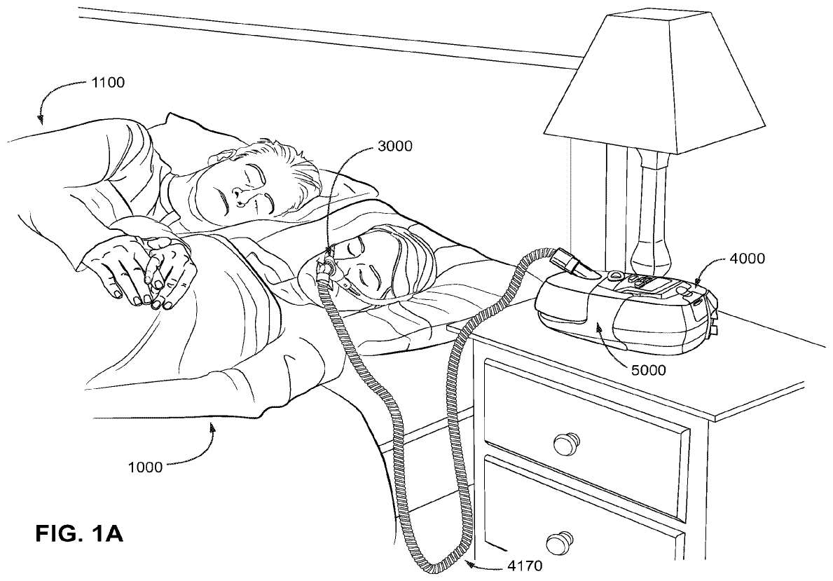

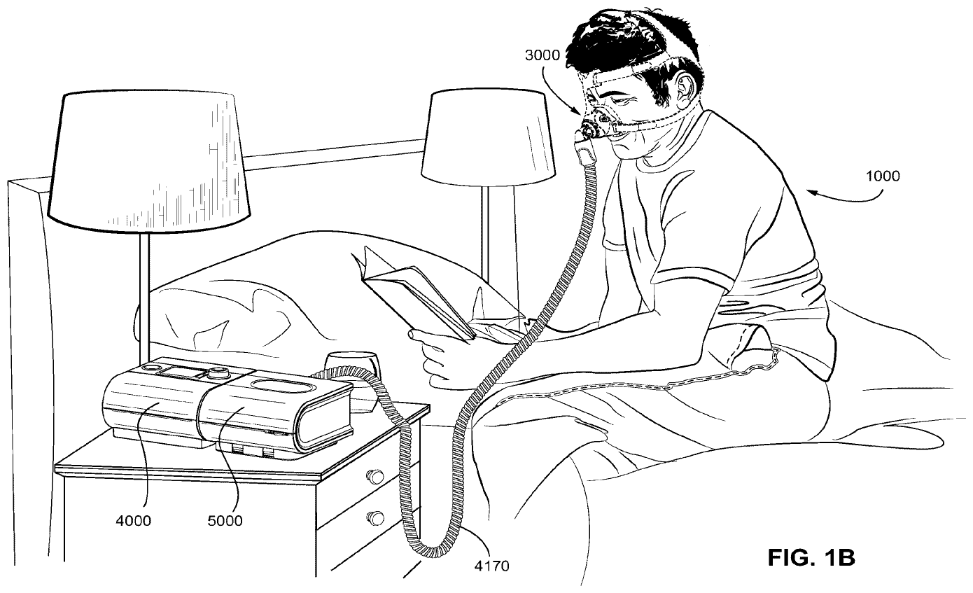

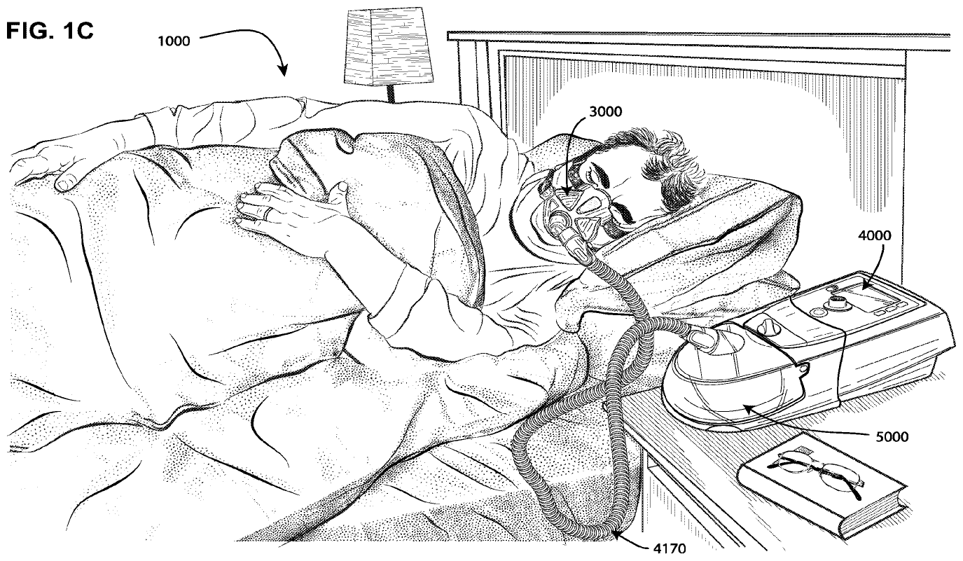

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

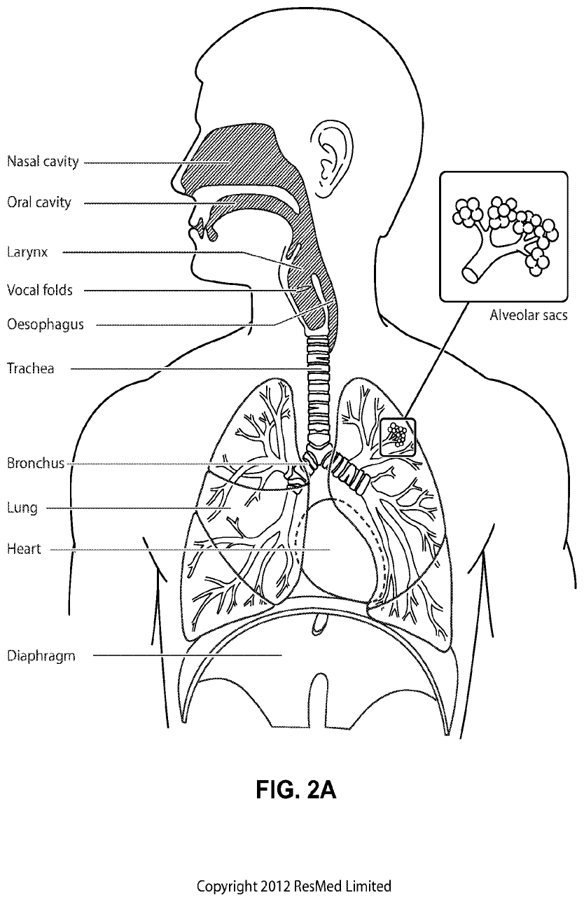

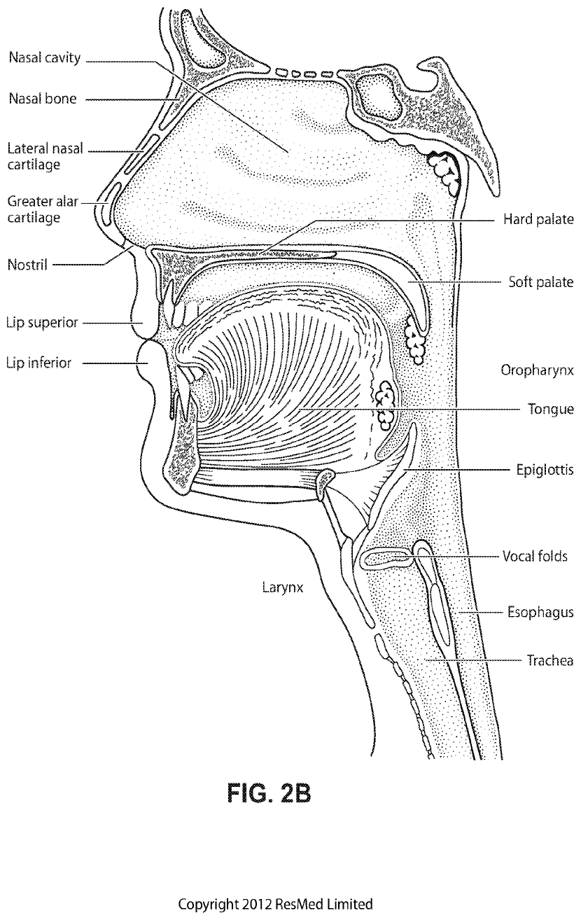

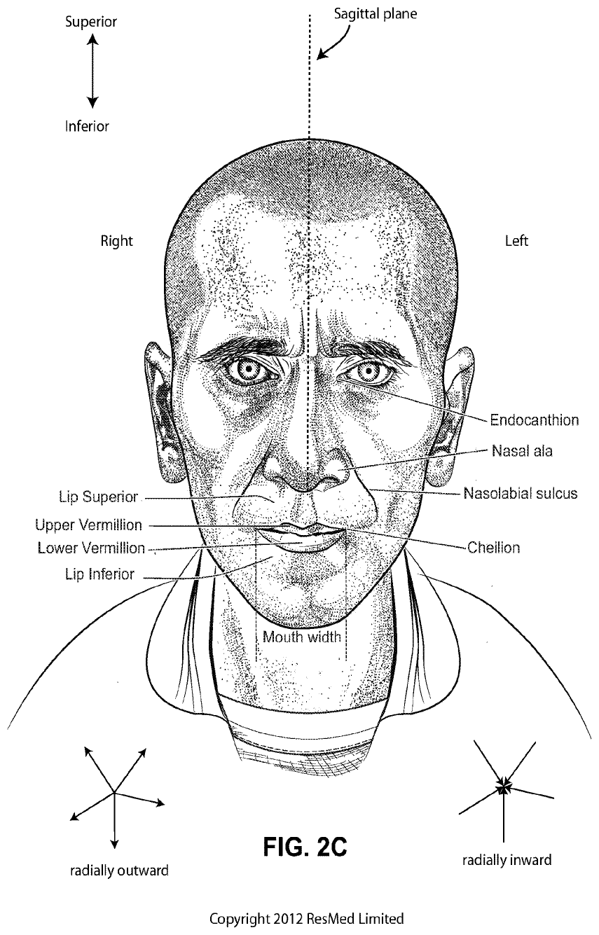

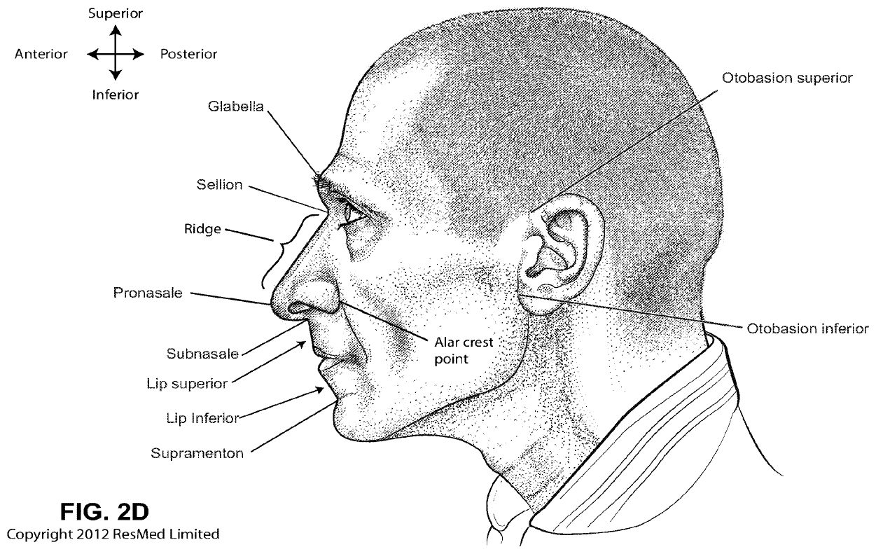

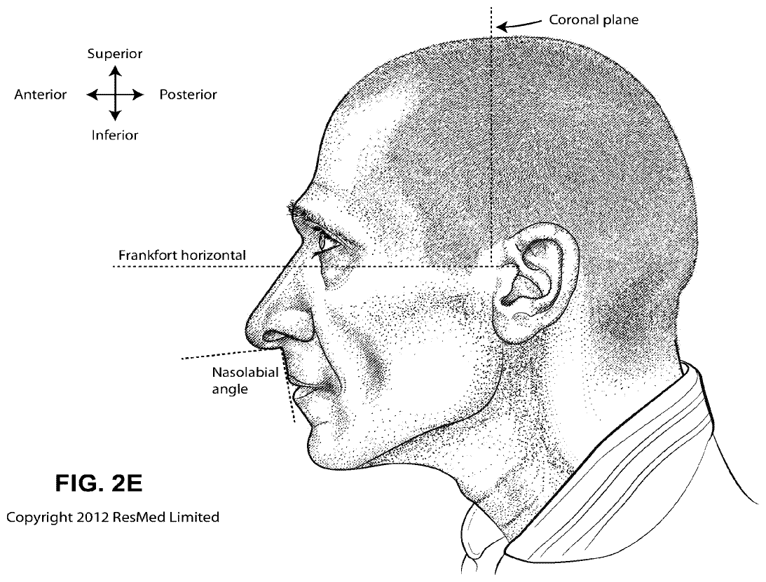

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

### 4.3 Patient Interface

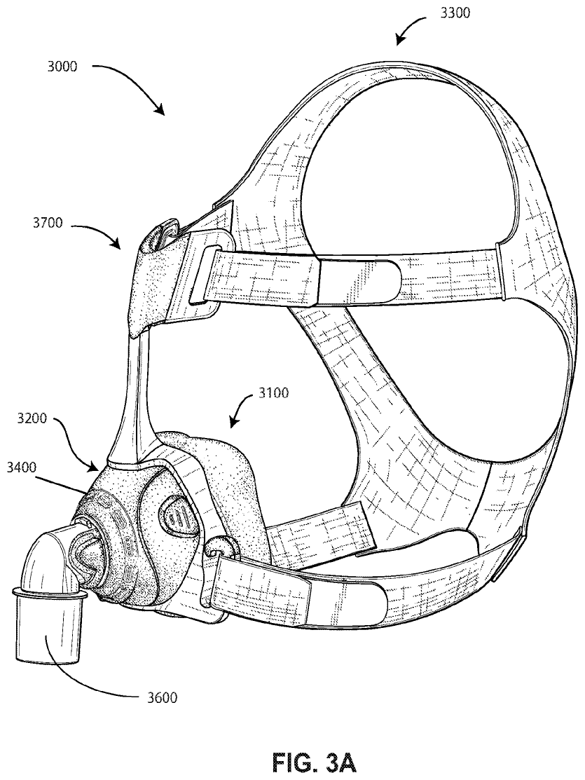

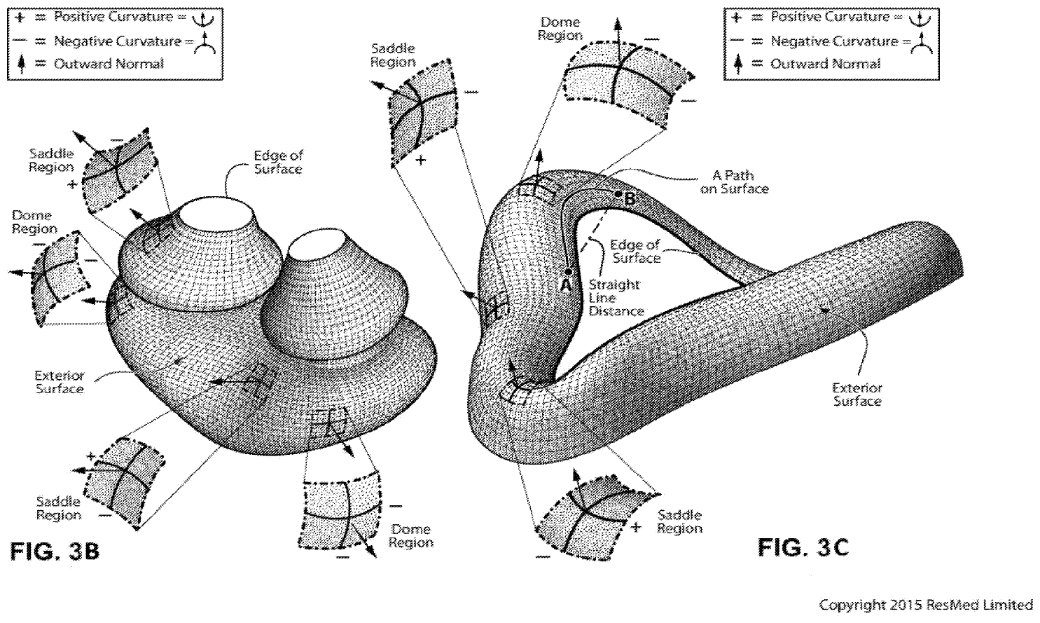

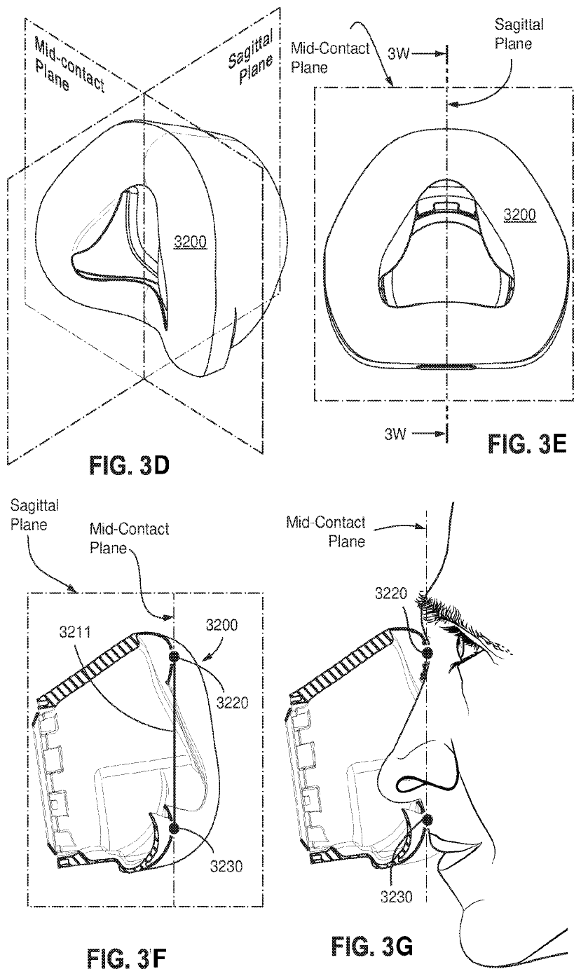

FIG. 3A shows a patient interface in the form of a nasal mask in accordance with one form of the present technology.

FIG. 3B shows a cushion for a mask that includes two pillows. An exterior surface of the cushion is indicated. An edge of the surface is indicated. Dome and saddle regions are indicated.

FIG. 3C shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3D shows a view of a plenum chamber 3200 showing a sagittal plane and a mid-contact plane.

FIG. 3E shows a view of a posterior of the plenum chamber of FIG. 3D.

The direction of the view is normal to the mid-contact plane. The sagittal plane in FIG. 3E bisects the plenum chamber into left-hand and right-hand sides.

FIG. 3F shows a cross-section through the plenum chamber of FIG. 3E, the cross-section being taken at the sagittal plane shown in FIG. 3E. A ‘mid-contact’ plane is shown. The mid-contact plane is perpendicular to the sagittal plane. The orientation of the mid-contact plane corresponds to the orientation of a chord 3211 which lies on the sagittal plane and just touches the cushion at two points on the sagittal plane: a superior point 3220 and an inferior point 3230. Depending on the geometry of the cushion in this region, the mid-contact plane may be a tangent at both the superior and inferior points.

FIG. 3G shows the plenum chamber 3200 of FIG. 3D in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3G the plenum chamber 3200 is that of a nasal mask, and the superior point 3220 sits approximately on the sellion, while the inferior point 3230 sits on the lip superior.

### 4.4 RPT Device

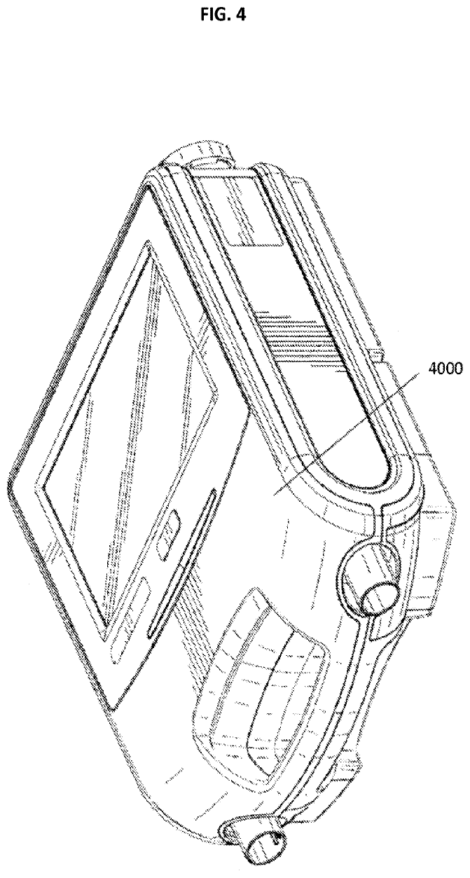

FIG. 4 shows an RPT device in accordance with one form of the present technology.

### 4.5 Cushion Modules

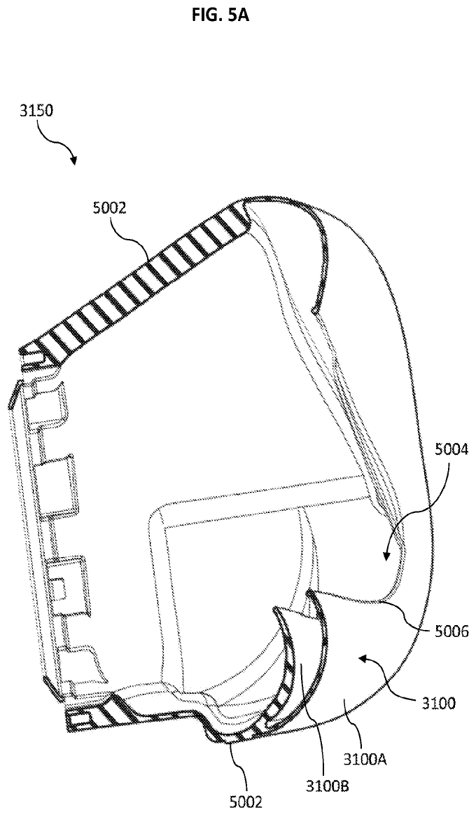

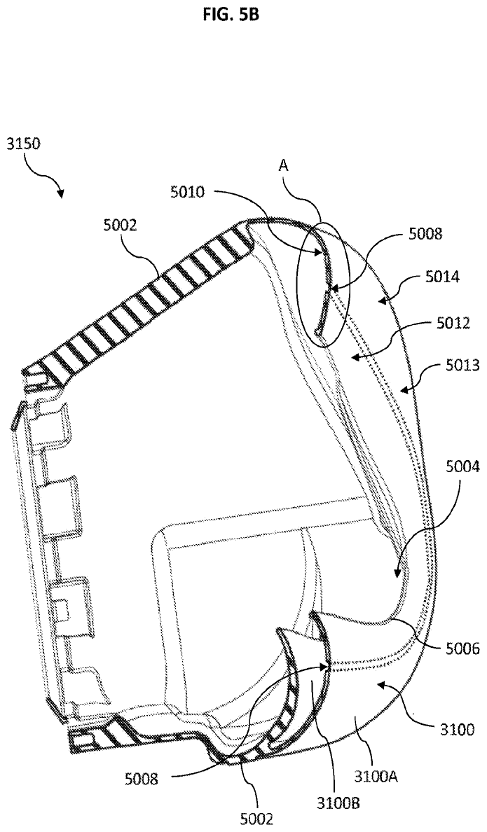

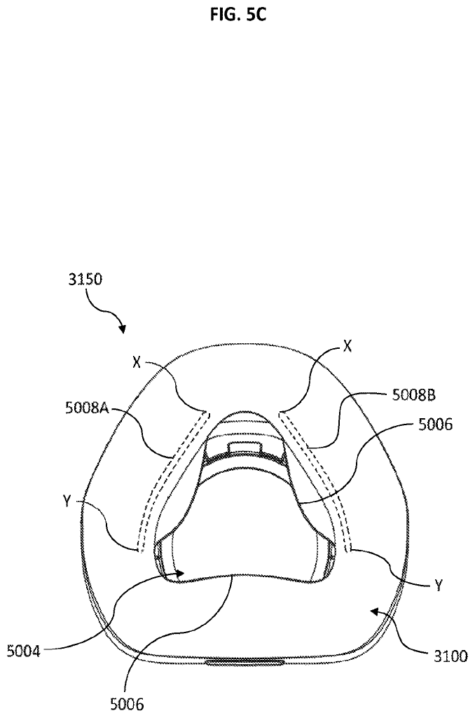

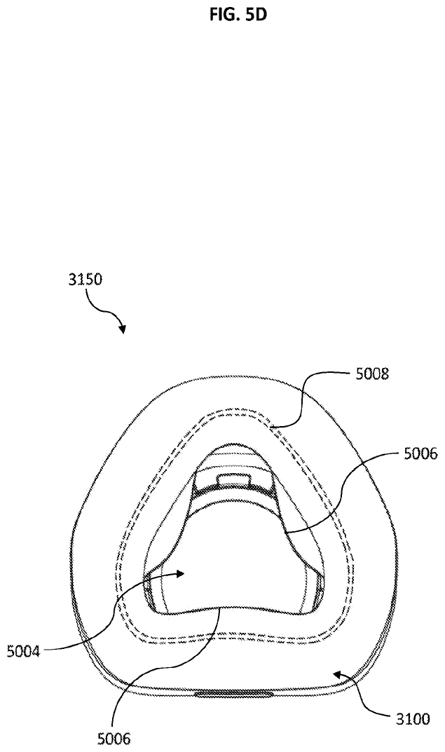

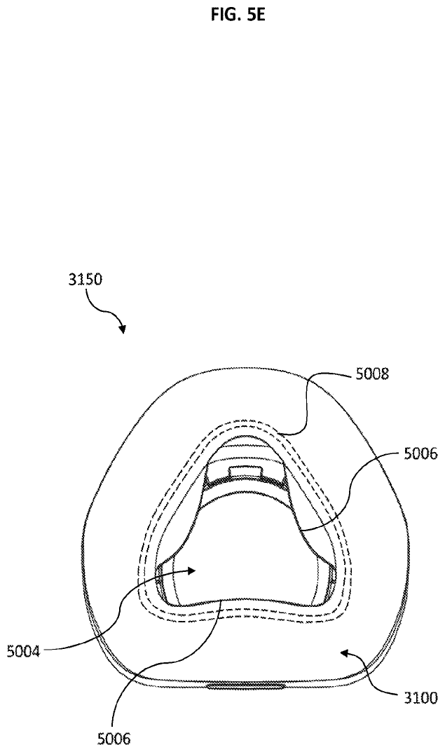

FIG. 5A shows a cross-sectional side view of a cushion module in accordance with one form of the present technology.

FIG. 5B shows a cross-sectional side view of a cushion module with a reduced thickness region in accordance with one form of the present technology.

FIG. 5C shows a rear view of a cushion module with a pair of reduced thickness region in accordance with one form of the present technology.

FIG. 5D shows an alternative rear view of a cushion module with a reduced thickness region in accordance with one form of the present technology.

FIG. 5E shows an alternative rear view of a cushion module with a reduced thickness region in accordance with one form of the present technology.

FIG. 5F shows an alternative rear view of a cushion module with a reduced thickness region in the patient contacting surface of the seal-forming structure in accordance with one form of the present technology.

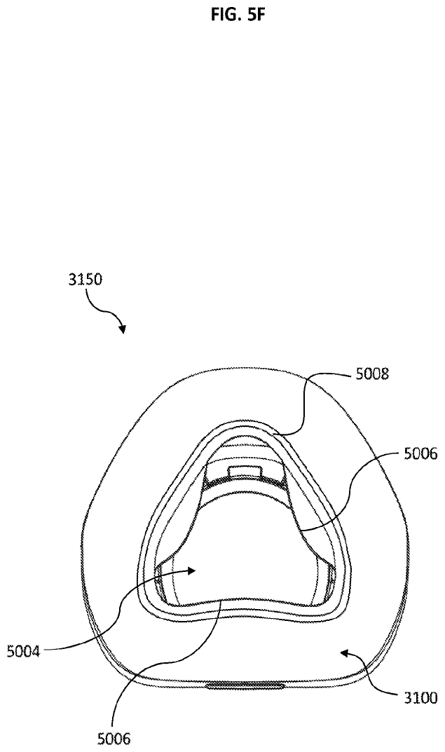

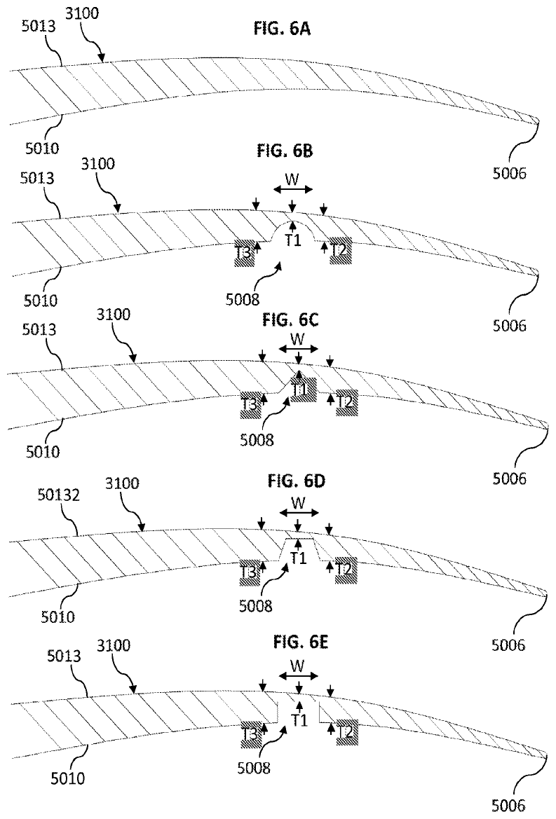

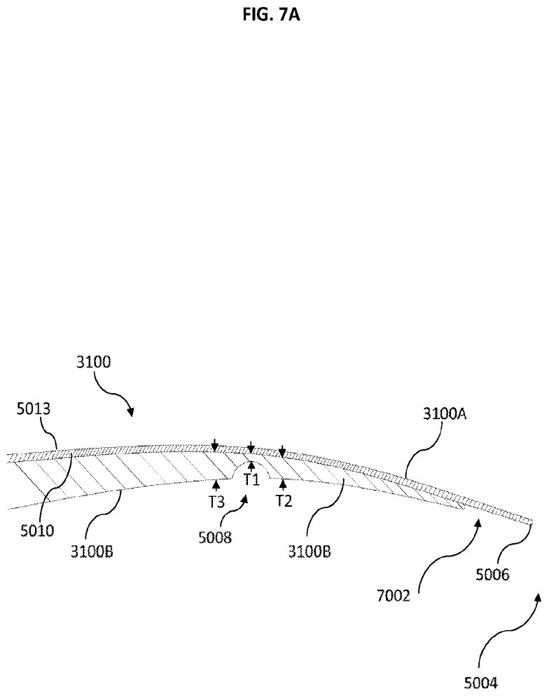

FIG. 6A shows a cross-sectioned view of a seal-forming structure in accordance with one form of the present technology.

FIG. 6B shows a cross-sectioned view of a seal-forming structure with a substantially curved reduced thickness region in accordance with one form of the present technology.

FIG. 6C shows a cross-sectioned view of a seal-forming structure with a substantially triangular reduced thickness region in accordance with one form of the present technology.

FIG. 6D shows a cross-sectioned view of a seal-forming structure with a substantially trapezoidal reduced thickness region in accordance with one form of the present technology.

FIG. 6E shows a cross-sectioned view of a seal-forming structure with a substantially rectangular reduced thickness region in accordance with one form of the present technology.

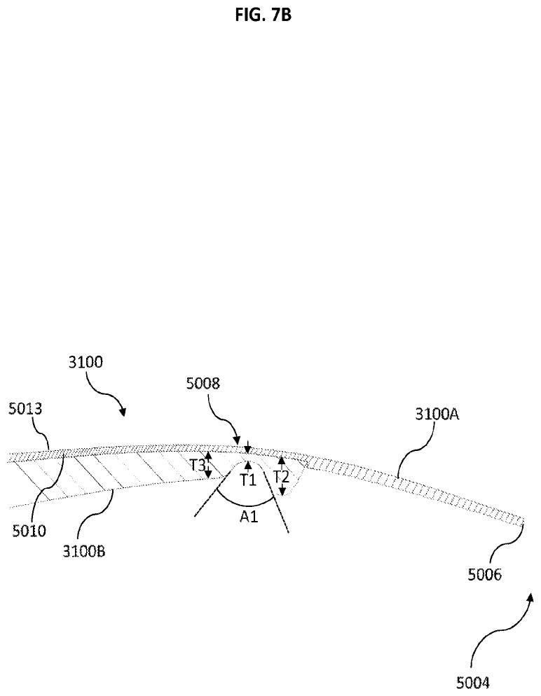

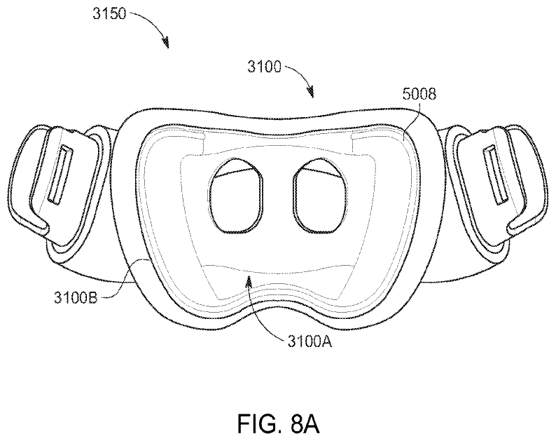

FIG. 7A shows a cross-sectioned view of a seal-forming structure comprising a patient contacting layer and a support layer in accordance with one form of the present technology.

FIG. 7B shows a cross-sectioned view of a seal-forming structure comprising a patient contacting layer and a support layer in accordance with another form of the present technology.

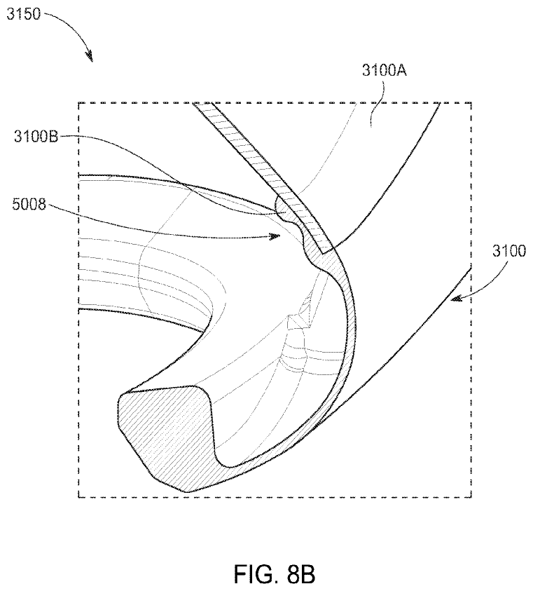

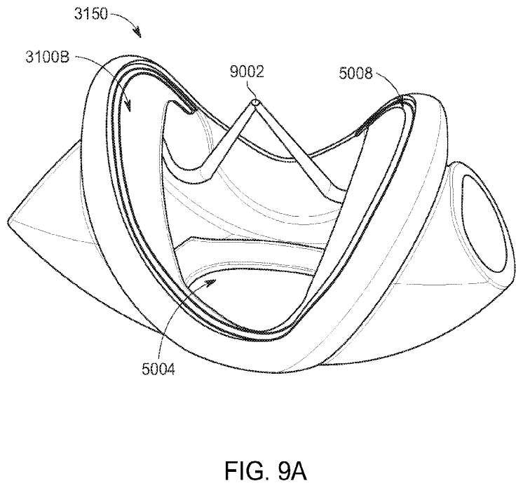

FIG. 8A shows a rear view of a cushion module comprising a textile patient contacting layer and a silicone support layer in accordance with one form of the present technology.

FIG. 8B shows a cross-sectional view of the cushion module of FIG. 8A showing the reduced thickness region in the silicone support layer.

### 4.6 Cushion Manufacturing

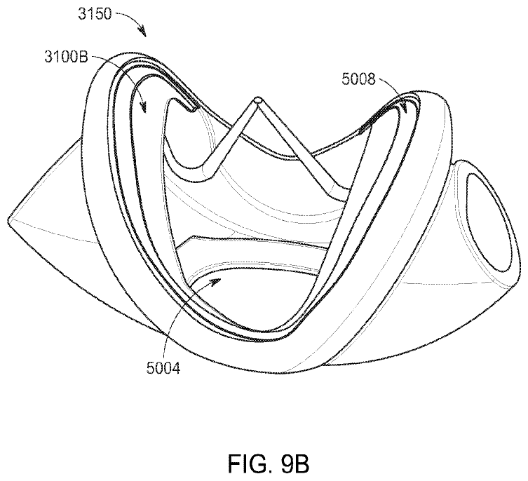

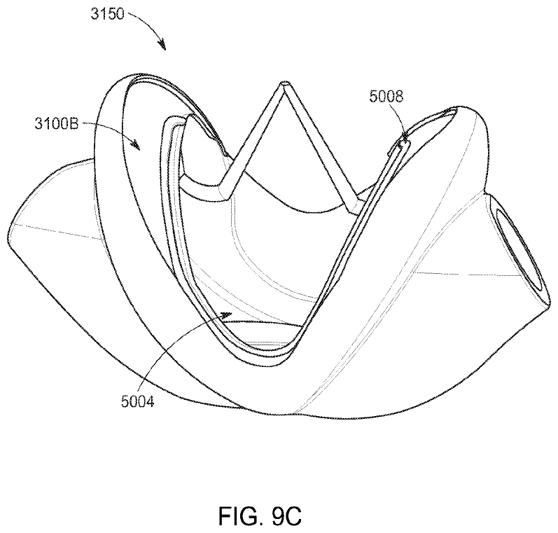

FIG. 9A shows a perspective view of a cushion module with an exemplary reduced thickness region and sprue locations in accordance with one form of the present technology.

FIG. 9B shows a perspective view of an alternative cushion module with an exemplary reduced thickness region and sprue locations in accordance with one form of the present technology.

FIG. 9C shows a perspective view of an alternative cushion module with an exemplary reduced thickness region and sprue locations in accordance with one form of the present technology.

### 4.7 Composite Cushion Module Structure

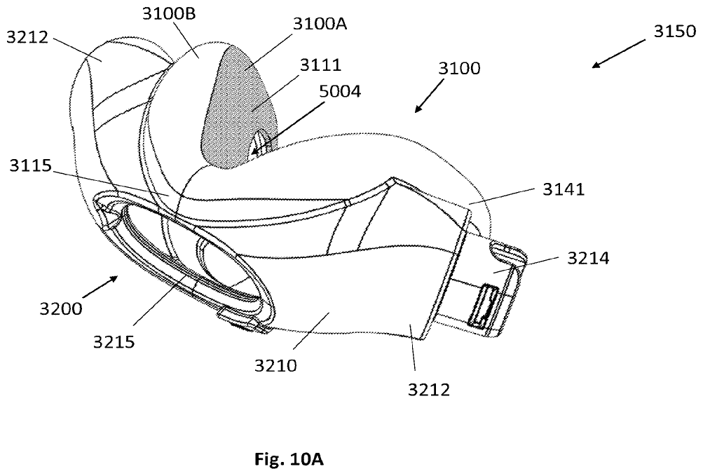

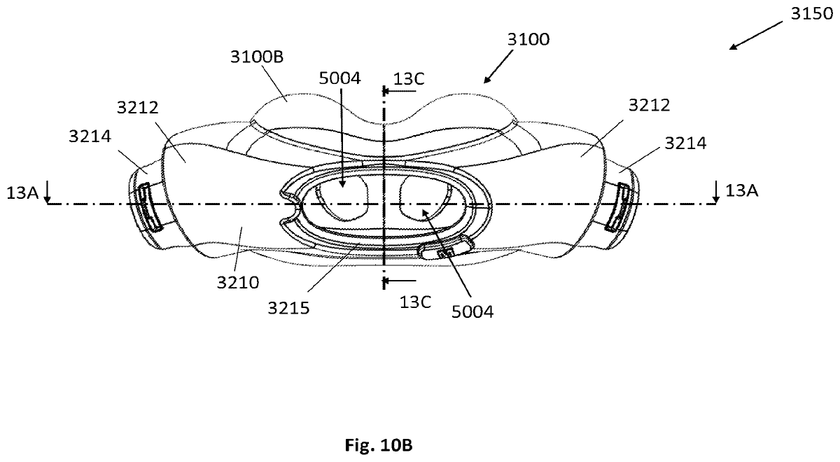

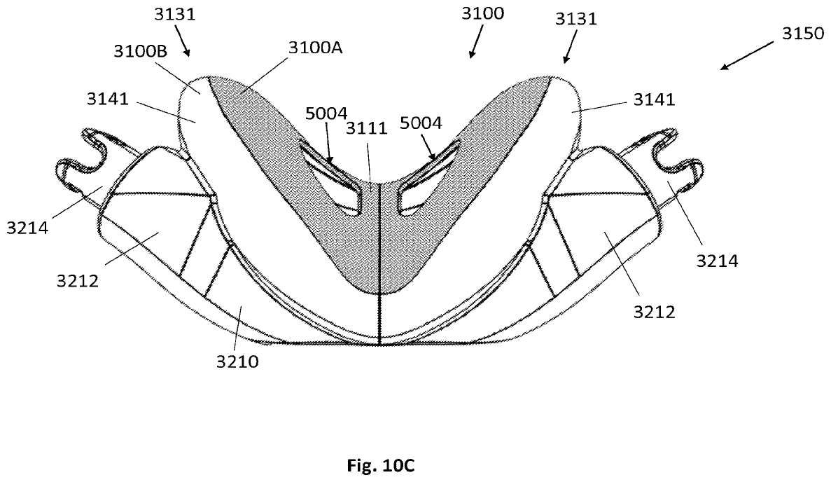

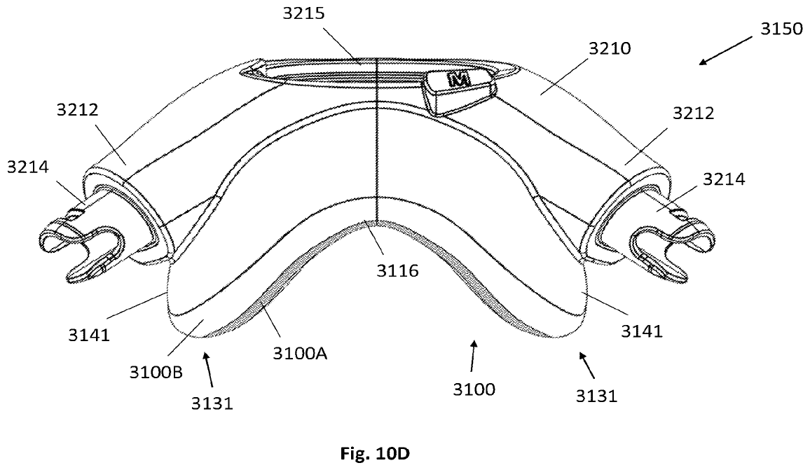

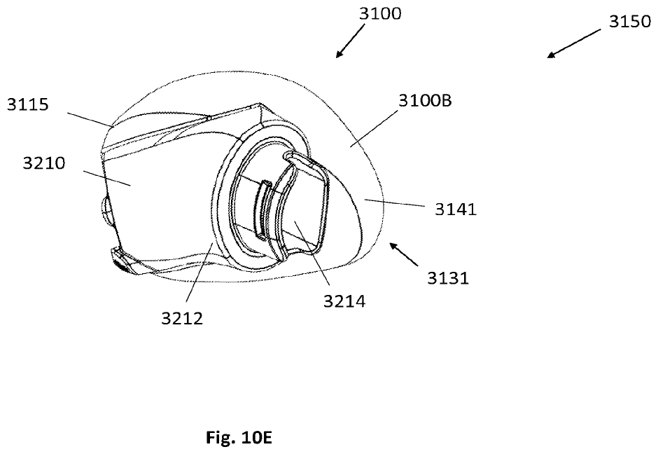

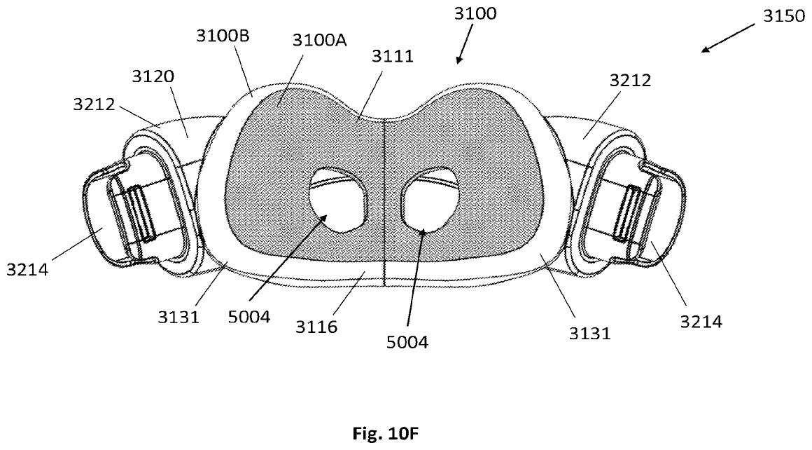

FIG. 10A shows a perspective view of a cushion module according to another example of the present technology.

FIG. 10B shows an anterior view of the cushion module shown in FIG. 10A.

FIG. 10C shows a superior view of the cushion module shown in FIG. 10A.

FIG. 10D shows an inferior view of the cushion module shown in FIG. 10A.

FIG. 10E shows a lateral view of the cushion module shown in FIG. 10A.

FIG. 10F shows a posterior view of the cushion module shown in FIG. 10A.

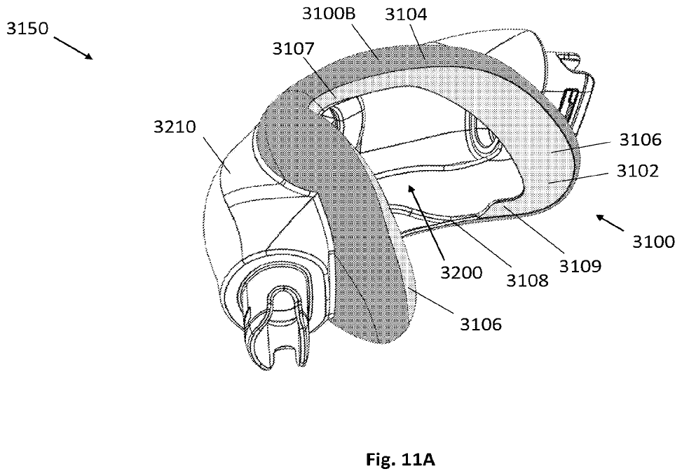

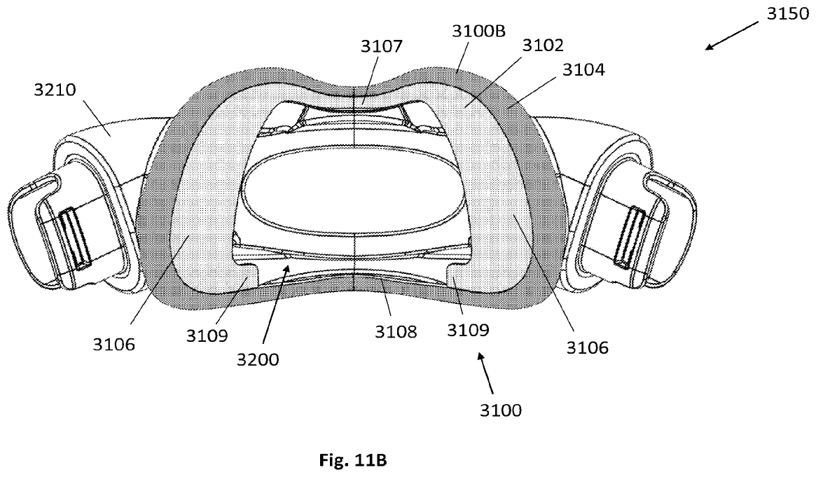

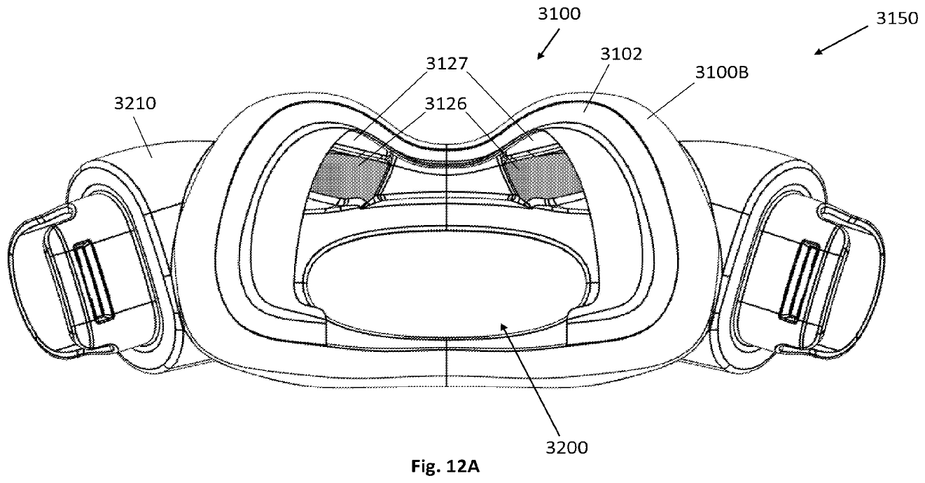

FIG. 11A shows a perspective view of the cushion module shown in FIG. 10A with a first layer of a seal-forming structure of the cushion module removed.

FIG. 11B shows a posterior view of the cushion module shown in FIG. 10A with the first layer of the seal-forming structure of the cushion module removed.

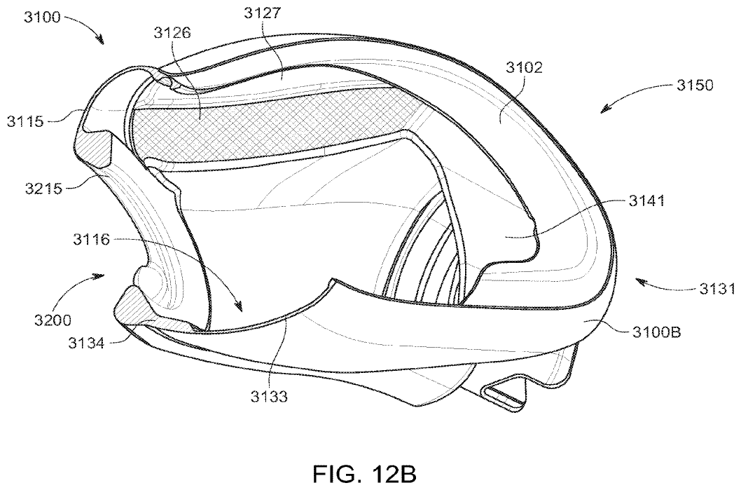

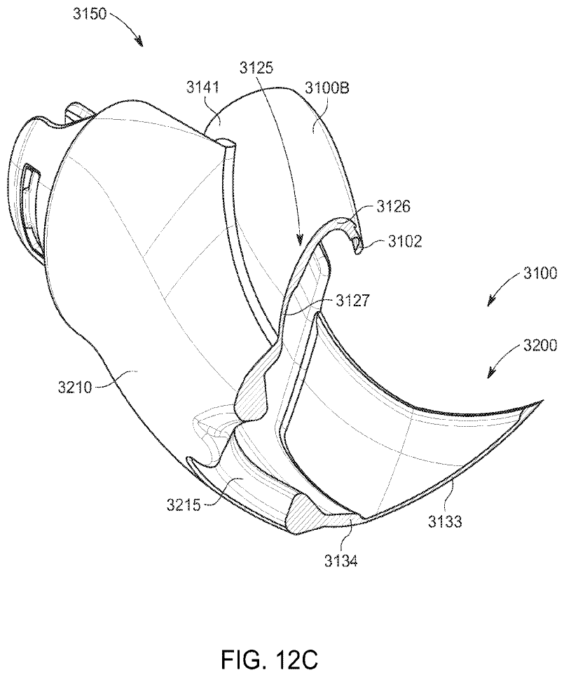

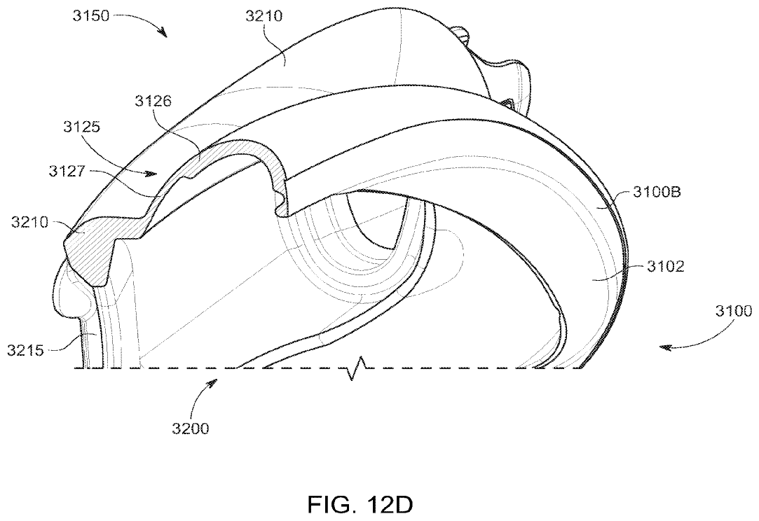

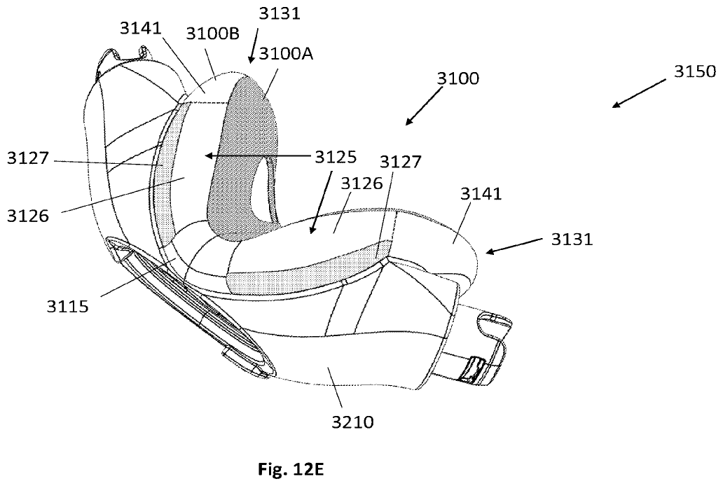

FIG. 12A shows a posterior view of the cushion module shown in FIG. 10A with the first layer of the seal-forming structure of the cushion module removed and with inferior portions of mid-lateral anterior portions of the seal-forming structure shaded.

FIG. 12B shows a cutaway view of the cushion module shown in FIG. 10A with the first layer of the seal-forming structure of the cushion module removed and with an inferior portion of a mid-lateral anterior portion of the seal-forming structure shaded.

FIG. 12C shows a cutaway cross section view of the cushion module shown in FIG. 10A with the first layer of the seal-forming structure of the cushion module removed.

FIG. 12D shows an alternative cutaway cross section view of the cushion module shown in FIG. 10A with the first layer of the seal-forming structure of the cushion module removed.

FIG. 12E shows a perspective view of the cushion module shown in FIG. 10A with the inferior portions of the mid-lateral anterior portions shaded.

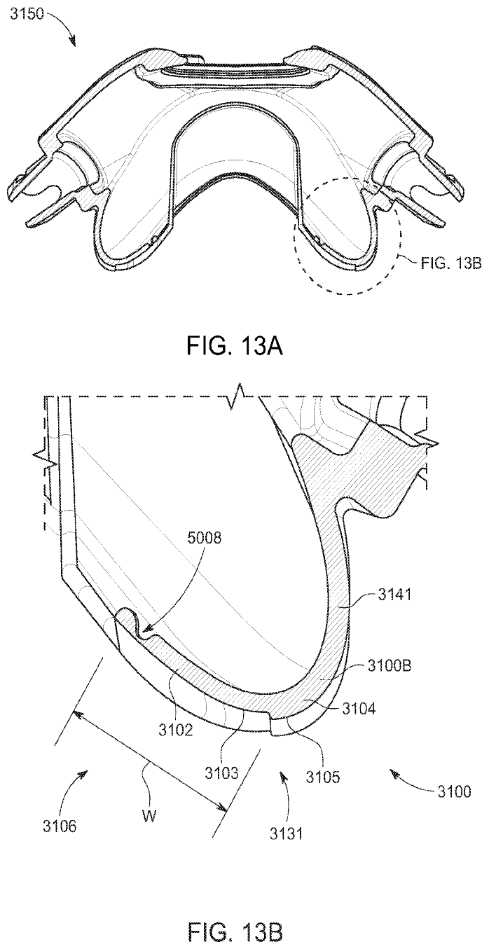

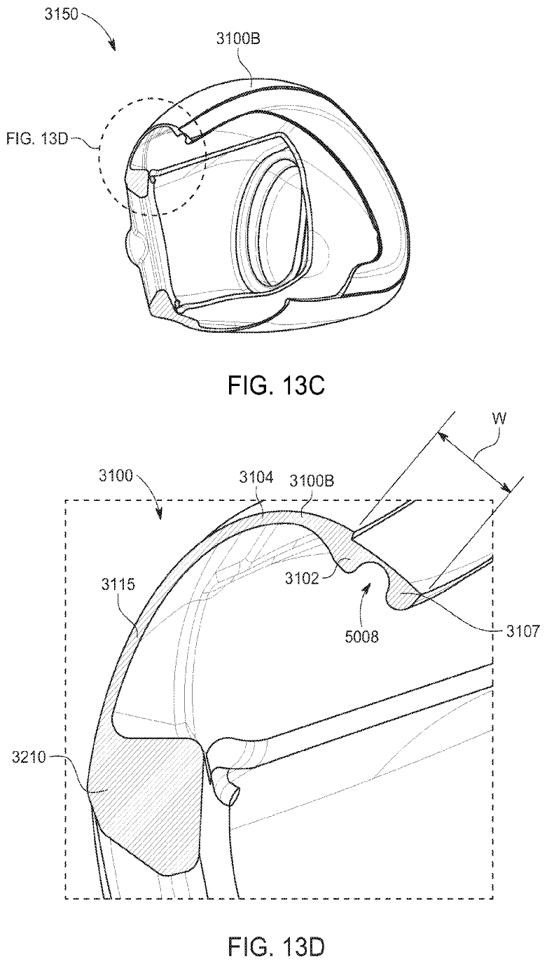

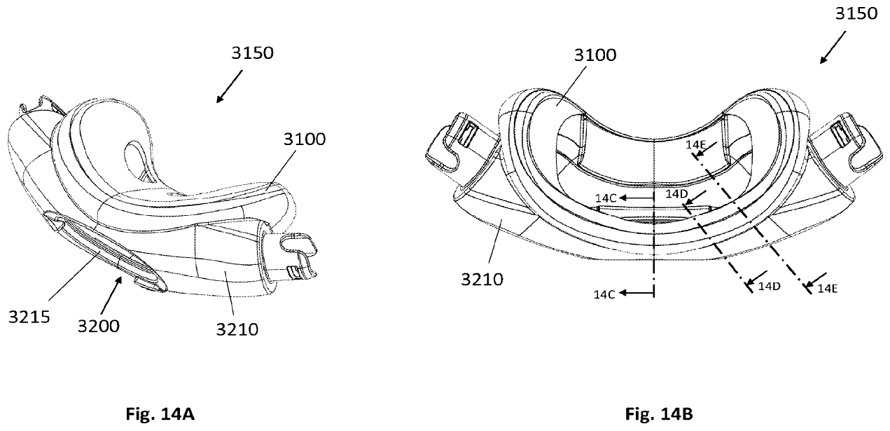

FIG. 13A is a cross section view of the cushion module shown in FIG. 10A through the cut 13A-13A marked in FIG. 10B.

FIG. 13B is a detail view of the area 13B indicated in FIG. 13A.

FIG. 13C is a cross section view of the cushion module shown in FIG. 10A through the cut 13C-13C marked in FIG. 10B.

FIG. 13D is a detail view of the area 13D indicated in FIG. 13C.

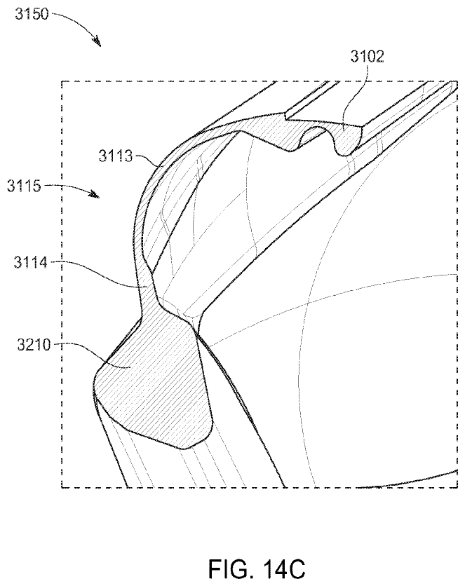

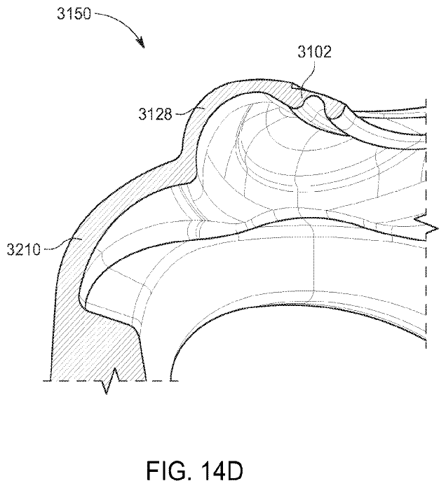

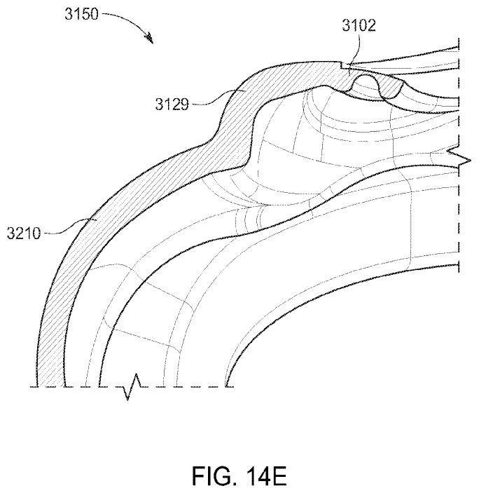

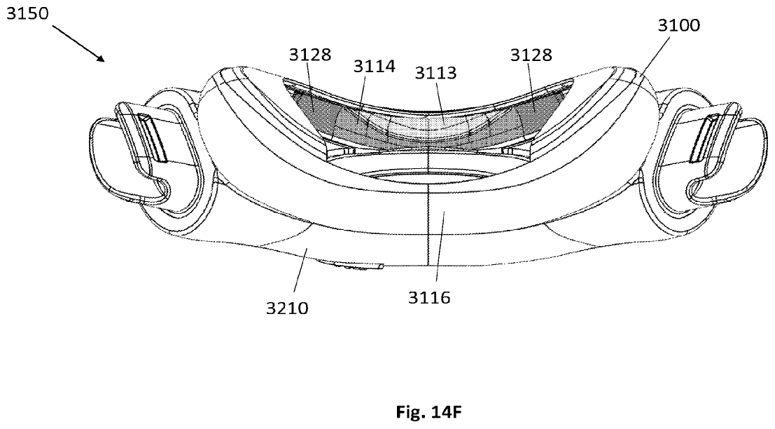

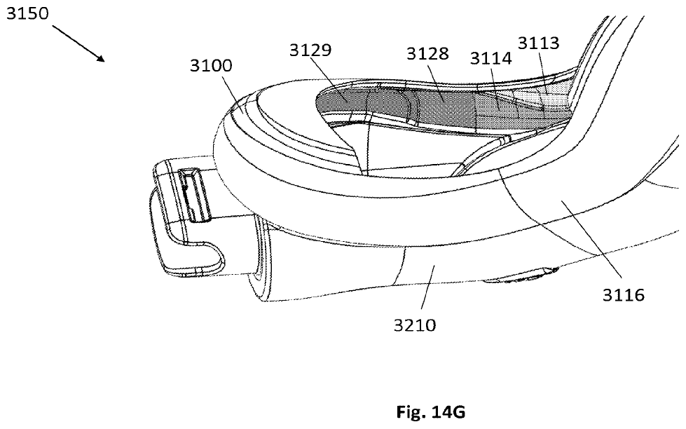

FIG. 14A is a perspective view of a cushion module according to another example of the present technology.

FIG. 14B is a top view of the cushion module shown in FIG. 14A with a first layer of the seal-forming structure removed.

FIG. 14C is a cross section view of the cushion module shown in FIG. 14A through the cut 14C-14C shown in FIG. 14B.

FIG. 14D is a cross section view of the cushion module shown in FIG. 14A through the cut 14D-14D shown in FIG. 14B.

FIG. 14E is a cross section view of the cushion module shown in FIG. 14A through the cut 14E-14E shown in FIG. 14B.

FIG. 14F is a posteroinferior view of the cushion module shown in FIG. 14A with certain portions of an anterior-facing wall of the seal-forming structure being shaded.

FIG. 14G is a lateral posteroinferior view of the cushion module shown in FIG. 14A with certain portions of an anterior-facing wall of the seal-forming structure being shaded.

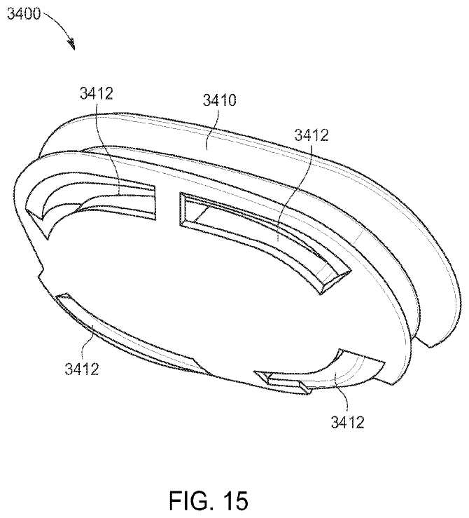

FIG. 15 is a perspective view of a vent module configured for use with the cushion module modules shown in FIG. 10A or 14A.

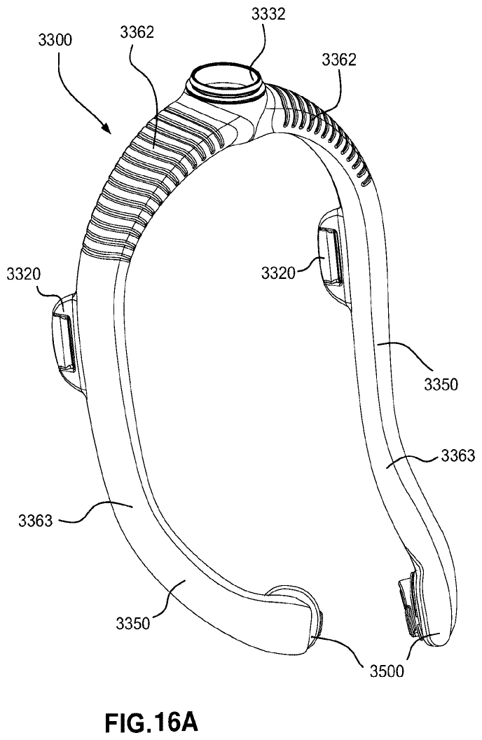

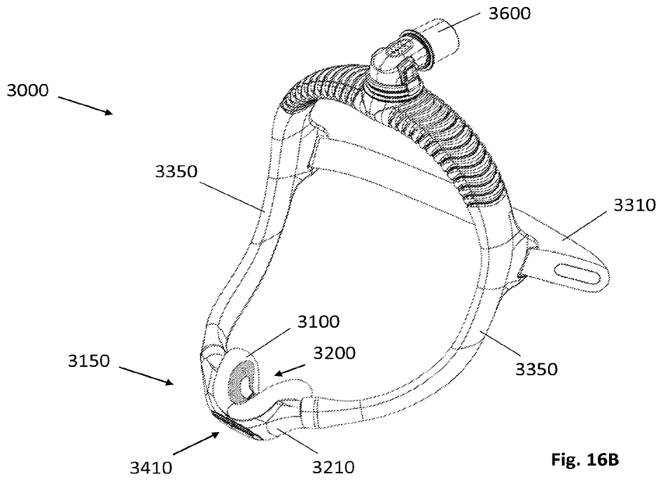

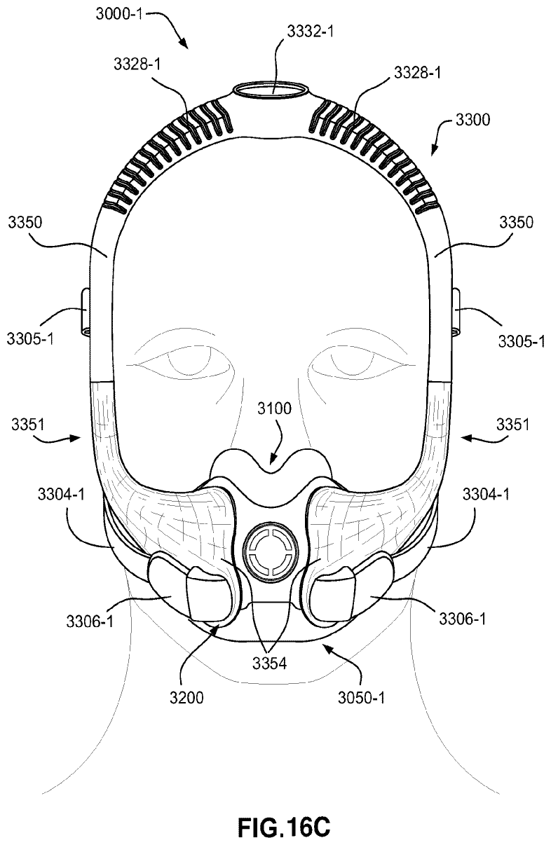

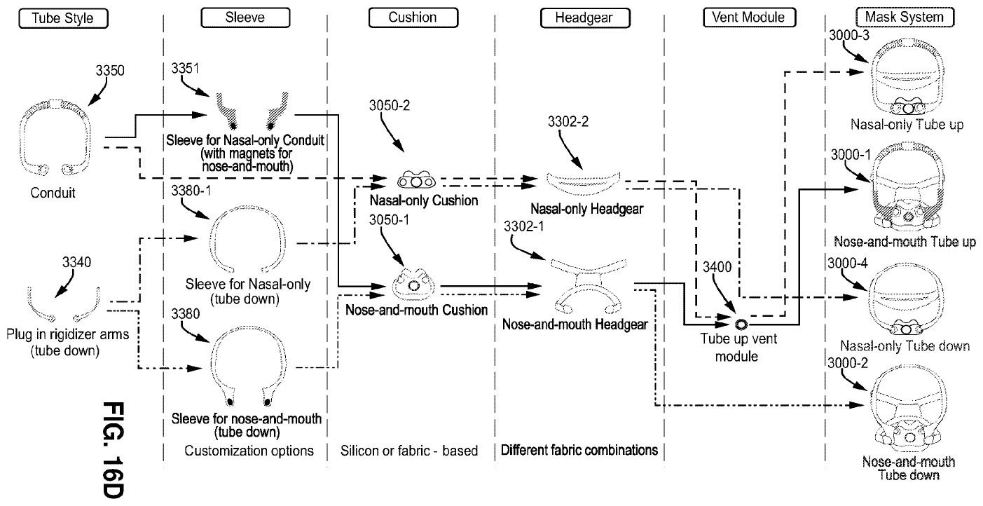

FIG. 16A shows a perspective view of exemplary gas delivery tubes usable with cushion modules.

FIG. 16B is a perspective view of a patient interface including the cushion module shown in FIG. 10A and gas delivery tubes shown in FIG. 16A.

FIG. 16C is perspective view of a patient interface comprising a nose and mouth cushion module with the gas delivery tubes shown in FIG. 16A.

FIG. 16D is a schematic view illustrating possible combinations of the patient interfaces.

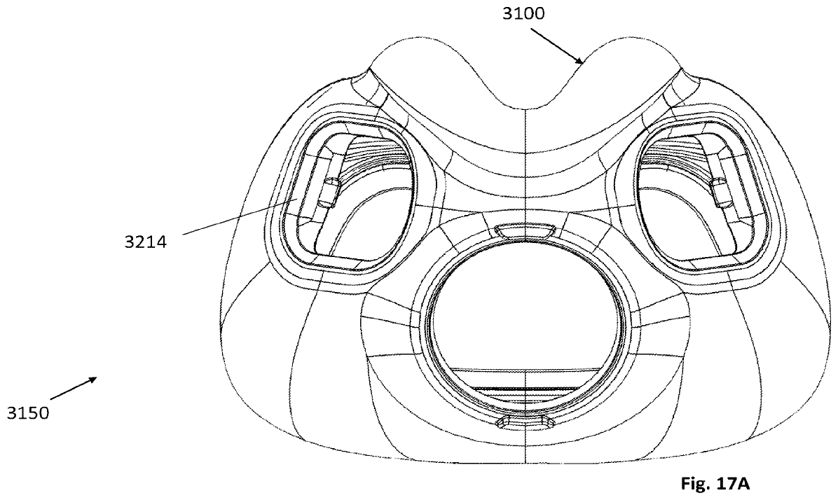

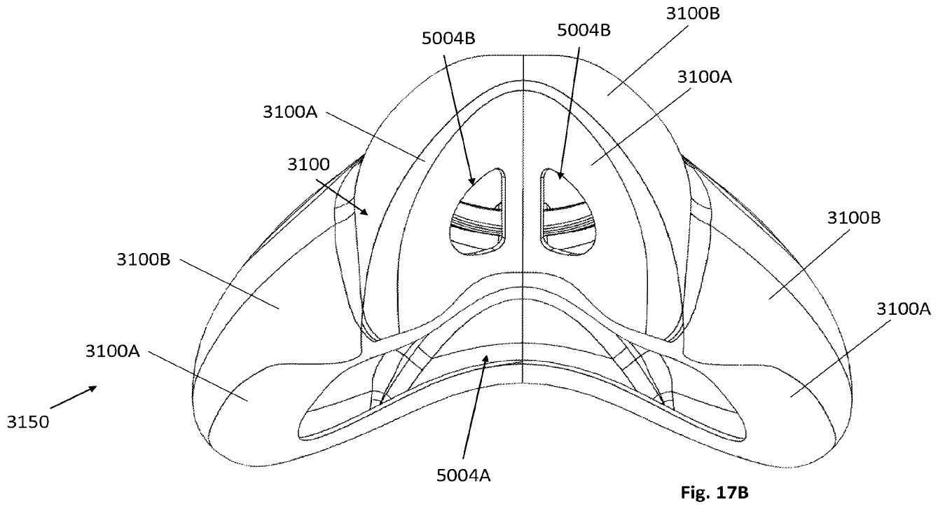

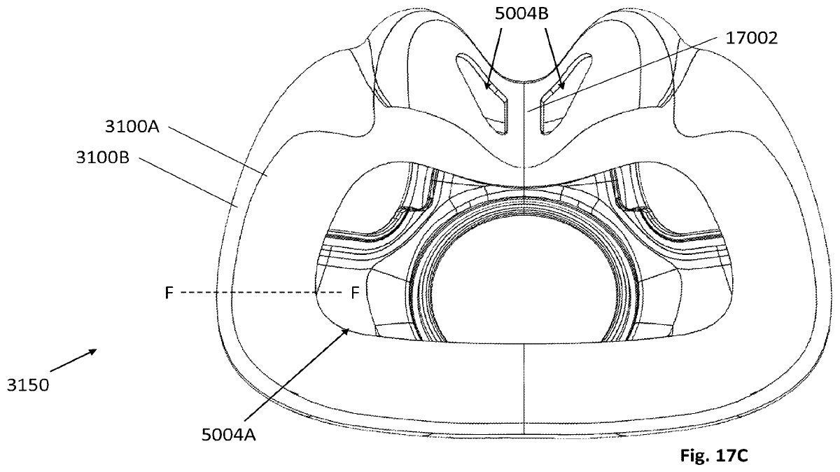

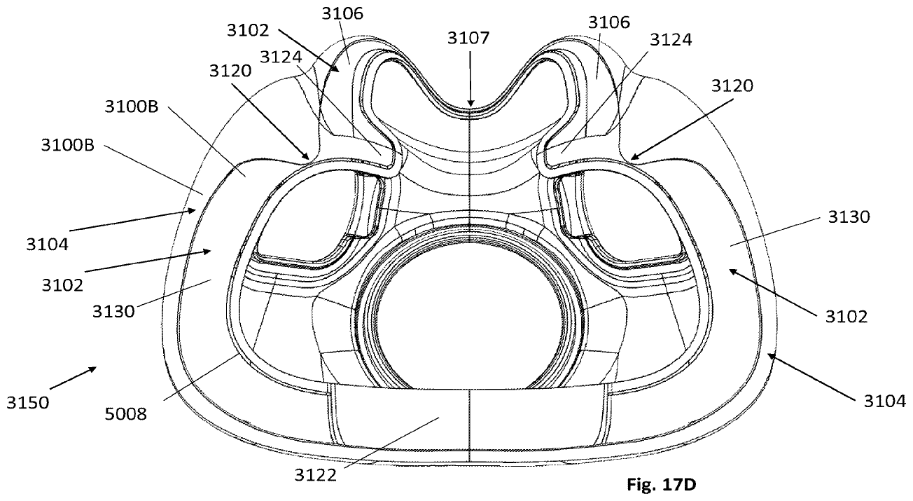

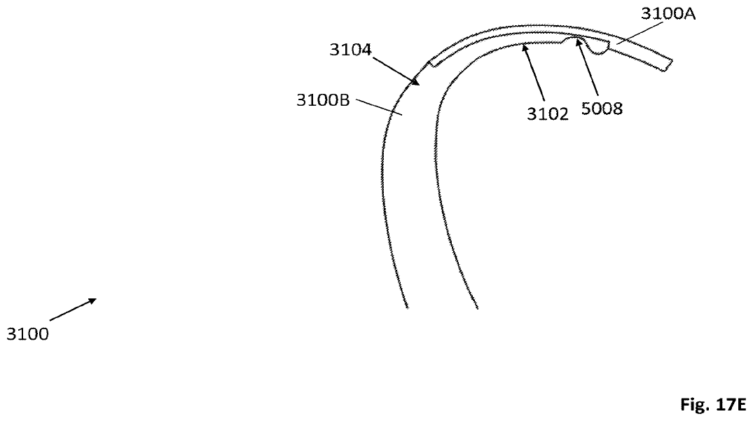

FIG. 17A is an anterior view of a nose and mouth cushion module according to another example of the present technology.

FIG. 17B is a posterior-superior view of the nose and mouth cushion module of FIG. 17A.

FIG. 17C is a posterior view of the nose and mouth cushion module of FIG. 17A.

FIG. 17D is a posterior view of the nose and mouth cushion module of FIG. 17A, with a first layer of the seal-forming structure removed.

FIG. 17E is a partial cross-sectional view through section line F-F of FIG. 17C.

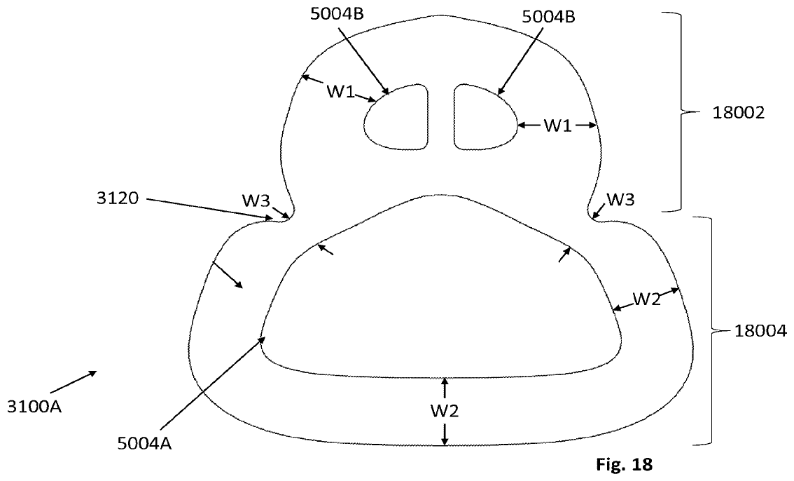

FIG. 18 is an anterior view of a first layer of a seal-forming structure according to another example of the technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient 1000 via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient 1000 so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient.

The patient interface 3000 in accordance with one form of the present technology is constructed and arranged to be able to provide a supply of air at a positive pressure of at least 2 cmH2O with respect to ambient.

The patient interface 3000 in accordance with one form of the present technology is constructed and arranged to be able to provide a supply of air at a positive pressure of at least 4 cmH2O with respect to ambient.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 5.3.1 Cushion

In one form of the present technology the patient interface comprises a cushion. The cushion may be a resiliently deformable structure configured to conform to the contours of the patient's face in use to provide a seal-forming structure 3100.

For example, the cushion may be constructed from a flexible material (e.g., constructed from a soft, flexible, resilient material like silicone, textile, foam, etc.). In some examples, the cushion may comprise a combination of materials, such as silicone and a textile, or silicone and a foam. In some forms of the technology, the cushion may be formed from a material which has a Young's modulus of 0.4 GPa or lower, for example foam. In some forms of the technology the cushion may be made from a material having Young's modulus of 0.1 Gpa or lower, for example rubber. In other forms of the technology the cushion may be made from a material having a Young's modulus of 0.7 Mpa or less, for example between 0.7 Mpa and 0.3 Mpa. An example of such a material is silicone.

In one form of the technology the cushion attaches to, or is moulded to, a rigid structure such as a chassis or other support structure in order to define, a cushion module 3150 which includes, at least in part a plenum chamber 3200. For example, the chassis may comprise a substantially rigid thermoplastic such as polycarbonate. In some examples the cushion is structured to at least in part, define the plenum chamber. For example, the cushion may be formed without a chassis, so as to substantially define the plenum chamber 3200.

The cushion may comprise an opening 5004 (see FIG. 5A for example) which is configured to convey the flow of breathable gas to the patient's airways in use. For example, in nose-only patient interfaces, the opening 5004 may be configured to receive the nose of the patient 1000 or otherwise seal around the nasal airways. In nasal pillows patient interfaces, the cushion may comprise one or more openings 5004 configured to provide the flow of breathable gas to the patient's airways. For example, a first opening 5004 may be configured to provide a flow of breathable gas to a first of the patient's nares and a second opening 5004 may be configured to provide a flow of breathable gas to a second of the patient's nares.

The opening 5004 may comprise a perimeter which defines an edge 5006 of the seal-forming structure 3100.

### 5.3.2 Cushion Module

The cushion may form part of a cushion module 3150, which may be a replaceable or non-replaceable component of the patient interface 3000. Cushion modules 3150 may be provided in different sizes, each usable as part of the patient interface 3000 such that the patient 1000 or their clinician is able to select the most appropriate size for the patient's face. A cushion module 3150 may comprise the cushion and other components or portions, such as a chassis or other support structure for the cushion, connectors and/or a vent module.

In some forms of the present technology, the cushion module 3150 is a portion of the patient interface 3000 forming the plenum chamber 3200 and seal-forming structure 3100. The cushion module 3150 may be separable from other components or portions of the patient interface 3000, such as a positioning and stabilising structure 3300, frame and/or connection port 3600, although in some examples the cushion module 3150 may not be separable from one or more other components of the patient interface 3000.

In one form of the present technology, shown in FIG. 5A the cushion module 3150 comprises a side-wall 5002 which extends substantially outwardly with respect to the patient's face in use, and a membrane or seal-forming structure 3100 which generally curves radially inwardly from the side-wall 5002 so as to form a seal with the patient's face in use.

In some examples, such as the example shown in FIGS. 10A-10F, the cushion module 3150 comprises a chassis portion 3210 and a seal-forming structure 3100. The seal-forming structure 3100 may be attached to or integrally formed (e.g. moulded in a single moulding step/shot) with the chassis portion 3210. The cushion module 3150 may also comprise other components, such as connectors and a vent module, in some examples. For example, the cushion module 3150 shown in FIGS. 10A-10F comprises a pair of connectors 3214 to connect to gas delivery tubes. The connectors 3214 are connected to the chassis portion 3210 of the cushion module 3150. The cushion module 3150 further comprises a vent module 3410 (shown separately in FIG. 15), configured to securely fit into an anterior hole 3215 of the chassis portion 3210. The vent module 3410 comprises a plurality of vent holes 3412 providing a vent 3400 for the patient interface 3000 for gas washout of the plenum chamber 3200 in use. The vent module may comprise a diffuser and may be removable for replacement or cleaning. In other examples of the present technology, the vent 3400 or vent module 3410 may take a different form. The chassis portion 3210 and the seal-forming structure 3100 of a cushion module 3150 may each partially form a plenum chamber 3200 of the patient interface 3000. The chassis portion 3210 may be stiffer than the seal-forming structure 3100 and may function as a chassis of the cushion module 3150. The chassis portion 3210 may provide support for the overall structure of the cushion module 3150 and seal-forming structure 3100. The chassis portion 3210 may hold an anterior or non-patient contacting side 5010 of the seal-forming structure 3100 in shape and position while a posterior or patient-contacting side 5013 of the seal-forming structure 3100 deforms to seal to the patient's face. The chassis portion 3210 and the seal-forming structure 3100 may together at least partially define a plenum chamber 3200 of the patient interface 3000.

Any feature of a cushion disclosed herein may be applied in a cushion module 3150 of a patient interface 3000. Any cushion module 3150 described herein may be provided with a chassis portion 3210 to support the seal-forming structure 3100.

In some examples, a cushion or cushion module 3150 may be attached to a frame in use. The cushion modules 3150 shown in FIGS. 5A-5F are of this type. The frame may form part of a positioning and stabilising structure 3300 and may provide connections to headgear straps. In some examples the cushion or cushion module 3150 may be connected directly to a positioning and stabilising structure 3300 without a frame, for example directly to headgear straps of a positioning and stabilising structure 3300 or gas delivery tubes of a positioning and stabilising structure 3300 that both holds the cushion module 3150 in place and also provides a pressurised flow of air or breathable gas to the interior of the cushion module 3150 (e.g. to the plenum chamber 3200), or both. The cushion modules 3150 shown in FIGS. 10A-10F and 17A-17D, for example, are of this type.

Any cushion, cushion module 3150, or seal-forming structure 3100 described herein, or a feature thereof, may be incorporated into a patient interface 3000 such as a patient interface 3000 in the form shown in FIG. 16B or in FIG. 16C.

### 5.3.3 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example, the system may comprise one form of a seal-forming structure 3100 suitable for a large sized head, but not a small sized head and another suitable for a small sized head, but not a large sized head.

### 5.3.3.1 Sealing Mechanisms

In one form, the seal-forming structure includes a sealing flange or patient contacting layer 3100A utilizing a pressure assisted sealing mechanism. In use, the sealing flange 3100A can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange or patient contacting layer 3100A and a support flange/layer 3100B (see FIGS. 5A-5C). The patient contacting layer 3100A comprises a relatively thin member with a thickness of less than about 1 mm, for example about 0.25 mm to about 0.45 mm, which extends around the perimeter of the plenum chamber 3200. Support layer 3100B may be relatively thicker than the patient contacting layer 3100A. The support layer 3100B is disposed between the patient contacting layer 3100A and the marginal edge of the plenum chamber 3200 and extends at least part of the way around the perimeter. The support layer 3100B is or includes a spring-like element and functions to support the patient contacting layer 3100A from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the patient contacting layer.

In one form, the seal-forming structure comprises a region having a tacky or adhesive surface.

In certain forms of the present technology, a seal-forming structure may comprise one or more of a pressure-assisted patient contacting layer, a compression sealing portion, a gasket sealing portion, a tension portion, and a portion having a tacky or adhesive surface.

### 5.3.3.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

For example, the seal-forming structure may comprise a sealing flange in the nasal bridge or nose-ridge of the patient's face.

### 5.3.3.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

For example, the seal-forming structure may comprise a sealing flange in the upper lip region of the patient's face.

### 5.3.3.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

For example, the seal-forming structure may comprise a sealing flange in the chin region of the patient's face.

### 5.3.3.5 Forehead Region

In one form, the seal-forming structure that forms a seal in use on a forehead region of the patient's face. In such a form, the plenum chamber may cover the eyes in use.

For example, the seal-forming structure may comprise a sealing flange in the forehead region of the patient's face.

### 5.3.3.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient 1000.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.3.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface 3000 may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening 5004 into two orifices, each of which, in use, supplies air or breathable gas to a respective one of the patient's nares. The bridge portion may be configured to contact or seal against the patient's columella in use. Alternatively, the seal-forming structure 3100 may comprise a single opening 5004 to provide a flow or air or breathable gas to both of the patient's nares.

In some forms, a nose-only mask may comprise nasal pillows, described above.

### 5.3.3.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose and mouth mask according to the present technology is what has traditionally been identified as a “full-face mask”, having a seal-forming structure 3100 configured to seal on the patient's face around the nose, below the mouth and over the bridge of the nose. A full-face mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening 5004 configured to deliver a flow of air or breathable gas to both nares and mouth of a patient 1000, may have an oral opening 5004A configured to provide air or breathable gas to the mouth and a nasal opening 5004B configured to provide air or breathable gas to the nares, or may have an oral opening 5004A for delivering air to the patient's mouth and two nasal openings 5004B for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.4 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient 1000 in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 comprises a transparent material, e.g. a transparent polycarbonate. For example, the plenum chamber may comprise, at least in part, a transparent chassis, shell or support structure. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 comprises a translucent material. For example, the plenum chamber may comprise, at least in part, a translucent chassis, shell or support structure. The use of a translucent material can reduce the obtrusiveness of the patient interface, and help improve compliance with therapy.

In some forms, the plenum chamber 3200 comprises a rigid material such as polycarbonate. For example, the plenum chamber may comprise, at least in part a rigid chassis, shell or support structure. The rigid material may provide support to the seal-forming structure 3100.

In some forms, the plenum chamber 3200 comprises a flexible material (e.g., constructed from a soft, flexible, resilient material like silicone, textile, foam, etc.). For example, in examples then may be formed from a material which has a Young's modulus of 0.4 Gpa or lower, for example foam. In some forms of the technology the plenum chamber 3200 may be made from a material having Young's modulus of 0.1 Gpa or lower, for example rubber. In other forms of the technology the plenum chamber 3200 may be made from a material having a Young's modulus of 0.7 Mpa or less, for example between 0.7 Mpa and 0.3 Mpa. An example of such a material is silicone.

### 5.3.5 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIG. 3A.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient 1000 while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

### 5.3.5.1 Headgear Straps

In some forms, the positioning and stabilising structure 3300 may include headgear 3302 with at least one strap which may be worn by the patient 1000 in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidizers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

### 5.3.6 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient 1000 while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.7 Decoupling Structure(s)

In one form the patient interface 3000 includes at least one decoupling structure, for example, a swivel or a ball and socket.

### 5.3.8 Connection Port

Connection port 3600 allows for connection to the air circuit 4170.

### 5.3.9 Forehead Support

In one form, the patient interface 3000 includes a forehead support 3700.

### 5.3.10 Anti-Asphyxia Valve

In one form, the patient interface 3000 includes an anti-asphyxia valve.

### 5.3.11 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.12 Modularity

The cushions, cushion modules 3150, headgear, and sleeves may come in different styles, which may correspond to different uses (e.g., mouth breathing, nasal breathing, etc.). A patient 1000 or clinician may select certain combinations of cushions, headgear, and sleeves in order to optimize the effectiveness of the therapy and/or the individual patient's comfort.

In some forms, the different styles of cushions, cushion modules 3150, headgear, and sleeves may be used interchangeably with one another in order to form different combinations of patient interfaces as illustrated in FIG. 16D. This may be beneficial from a manufacturing perspective because wider variety of patient interfaces may be created using fewer parts. Additionally, or alternatively, the various combinations may allow a patient 1000 to change styles of patient interface without changing the every component.

As described above, the cushion may form part of a cushion module 3150, and multiple cushion modules 3150 having different sizes may be available for use with a particular positioning and stabilising structure 3300 to enable the patient interface 3000 to fit a wide range of patients 1000.

### 5.3.13 Cushion Design

FIG. 5A shows one example of a cushion module 3150 in accordance with the present technology comprising a seal-forming structure 3100. In the example of FIG. 5A the seal-forming structure 3100 comprises a supporting layer 3100B and patient contacting layer 3100A, however this should not be seen as limiting on the technology.

In some forms, the seal-forming structure 3100 of the cushion module 3150, may comprise a first region otherwise referred to as a thin or reduced thickness region 5008 as shown in FIG. 5B. In the illustrated example the reduced thickness region 5008 is provided in the seal-forming structure 3100 between the edge 5006 of the seal-forming structure 3100 and the side-wall 5002. In the illustrated example, the reduced thickness region 5008 is provided in a non-patient contacting side 5010 or anterior surface of the seal-forming structure 3100. Positioning the reduced thickness region 5008 in the non-patient contacting side 5010 of the seal-forming structure, or in a supporting layer 3100B of the seal-forming structure, may advantageously improve patient 1000 comfort. However, this should not be seen as limiting on the technology, and in other examples the reduced thickness region 5008 could alternatively be positioned in the patient contacting side 5013 of the seal-forming structure 3100.

With reference to FIGS. 3C and 5C, the reduced thickness region 5008 may follow a path on the surface of the seal-forming structure 3100 indicated by the curved line between points X and Y, or as a closed loop as shown in FIGS. 5D-5F. The path may extend in a direction which is substantially parallel to the edge 5006 of the seal-forming structure 3100, in a circumferential direction around at least a portion of the seal-forming structure 3100.

Reference herein to the circumferential direction should be understood to be defined with respect to a longitudinal axis extending through the centres of the front and rear openings 5004 of the cushion module 3150.

The reduced thickness region 5008 in the examples described herein is provided at a radial distance from the longitudinal axis. In examples the reduced thickness region(s) 5008 extend around a portion of the seal-forming structure 3100 of the patient interface 3000 in a circumferential direction, in other words between a first point X, and a second point Y, wherein the magnitude of the angle between points X and Y when measured with respect to the longitudinal axis is greater than 0, or more preferably greater than 30 degrees or greater than 0.52 radians. In some examples of the technology such as the examples of FIGS. 5D-5F, the reduced thickness region 5008 forms a closed loop around the seal-forming structure 3100, or otherwise extends through 360 degrees or 2 pi radians with respect to the longitudinal axis. It is to be understood that while the terms circumferential and radial are used to assist in describing directions, the reduced thickness region 5008 and/or other components of a seal-forming structure 3100 or cushion module 3150 may not be circular in practice.

As the reduced thickness region 5008 extends in a circumferential direction around the surface of the seal-forming structure 3100, the radial distance from the longitudinal axis may vary, for example to define a non-circular path, to follow the contour of the seal-forming structure 3100, or otherwise to maintain a substantially constant distance from the edge 5006 of the seal-forming structure 3100 in one or more regions, such as the nasal bridge region, side of nose regions, upper lip, or chin regions of the seal-forming structure 3100.

Throughout the present specification, reference to a thin or reduced thickness region 5008 on the seal-forming structure 3100 or cushion module 3150 should be understood to include a region which comprises a thickness which is less than one or more adjacent regions. For example, the seal-forming structure 3100 may transition from a first region having a first thickness to a second region having a second thickness, the second thickness being less than the first thickness. In some forms of the technology, the transition from the first region to the second region may occur in a direction between the inner edge 5006 and a side-wall 5002 of the forming structure 3100, however in other examples the transition may be provided in any direction including for example around the perimeter of the seal-forming structure 3100.

In some examples of the technology the seal-forming structure 3100 may comprise a plurality of first and second regions, for example as shown in FIG. 5C. In examples the plurality of first and second regions are separate from one another, however this should not be seen as limiting. For example, two second regions (reduced thickness regions 5008A and 5008B) may be provided which are separate to each other, but contiguous with a single first region (e.g. the region surrounding the reduced thickness regions 5008A and 5008B) having a thickness greater than the second region.

In some forms the reduced thickness region 5008 substantially follows the contour of the seal-forming structure 3100, for example the reduced thickness region 5008 may have an elongate profile which substantially follows the profile of the seal-forming structure 3100, or otherwise follows the contour of the seal-forming structure 3100 in a circumferential direction or a direction which is substantially parallel to at least a portion of the inner perimeter of the patient contacting portion, such as the edge 5006.

In some forms of the technology the reduced thickness region 5008 has a thickness which is less than at least part of the seal-forming structure 3100 between the edge 5006 and the reduced thickness region 5008. In other words, the reduced thickness region 5008 may be positioned between an inner section 5012, of the seal-forming structure 3100, substantially adjacent to the opening 5004, and an outer section 5014 of the seal-forming structure 3100 substantially adjacent to the side-wall 5002.

The seal-forming structure 3100 may comprise a first region 5012 having a first thickness, and a second reduced thickness region 5008 which is contiguous with at least a portion of the first region, whereby the reduced thickness region 5008 has a thickness which is less than the first thickness. It should be appreciated that the thickness of the first region and/or reduced thickness regions 5008 may vary around the seal-forming structure 3100, such as around the opening 5004 in the seal-forming structure 3100. However, in accordance with one example of the present technology, the thickness of the reduced thickness region 5008 may be less than the thickness of the first region at least in the area of the first region immediately adjacent to the reduced thickness region 5008.

In some forms of the technology the reduced thickness region 5008 has a thickness which is less than another thickness with a portion of the seal-forming structure 3100 between the side-wall 5002 and the reduced thickness region 5008 (i.e. the outer section 5014). For example, the reduced thickness region 5008 may have a thickness which is less than at least part of the seal-forming structure 3100 between the edge 5006 and the reduced thickness region 5008, and at least part of the seal-forming structure between the side-wall 5002 and the reduced thickness region 5008.

In some examples of the technology, the reduced thickness region 5008 may be provided in the form of a channel which extends along one or more regions of the seal-forming structure 3100 of the cushion module 3150. For example, FIG. 5C shows one example of a patient interface comprising a first reduced thickness region 5008A and a second reduced thickness region 5008B. In the illustrated example the first reduced thickness region 5008A is provided in a first side-of-nose region of the seal-forming structure 3100, and the second reduced thickness region 5008B is provided in a second side-of-nose region of the seal-forming structure 3100. This example should not be seen as limiting and these reduced thickness regions 5008 can be provided in any region of the cushion module 3150, preferably within the seal-forming structure 3100 of the cushion module 3150 between the opening 5004 and side-wall 5002. For example, the reduced thickness regions 5008 can be provided in any one or more of: side-of-nose regions; a nasal bridge region; an upper lip region; cheek regions; a lower lip region; a chin region, a pronasale region and/or a forehead region of the patient interface 3000. For example, it may be advantageous to provide a reduced thickness region 5008 in the cheek, and chin regions of a full-face mask while avoiding the nasal bridge region of the seal-forming structure 3100.

In the example of FIG. 5C the first reduced thickness region 5008A is substantially symmetrical to (e.g. a mirror image of) the second reduced thickness region 5008B. Use of symmetrically arranged reduced thickness regions 5008 may be advantageous to match the substantially symmetrical facial features of the patient 1000 in use.

The reduced thickness regions 5008 may be positioned in any location on the cushion module 3150, preferably within the seal-forming structure 3100 or otherwise the surface of the cushion module 3150 between the edge 5006 and the side-wall 5002. In some examples it may be advantageous for the reduced thickness region(s) 5008 to be positioned adjacent to the edge 5006 of the seal-forming structure 3100 such as between 1 mm and 15 mm from the edge 5006, such as between 2 mm and 5 mm from the edge 5006.

In some examples of the technology, such as the example of FIG. 5D, the reduced thickness region 5008 may be provided as a continuous channel that extends in a circumferential direction around the seal-forming structure 3100, and forms a closed loop on the seal-forming structure 3100. For example, the reduced thickness region 5008 may follow the contour of the seal-forming surface of the cushion module 3150 in a circumferential direction around a full perimeter of the seal-forming structure 3100.

Use of one or more reduced thickness regions 5008 in the seal-forming structure 3100 can create fold points which can further reduce the amount of pressure being transferred to the soft tissues of the patient's face. For example, the reduced thickness region 5008 can act as a pivot point which allows for the section of the seal-forming surface between the opening 5004 and the reduced thickness region 5008 to pivot about the reduced thickness region 5008 more easily than if there was no reduced thickness.

The reduced thickness region 5008 can have any suitable geometry and can be positioned in any suitable region of the seal-forming structure 3100. For example, FIG. 5E shows a further alternative example of a patient interface 3000 in which the reduced thickness region 5008 is provided as a continuous channel or closed loop which is located more radially inward, or closer to the edge 5006 on the seal-forming structure 3100 than the example of FIG. 5D. For example, the reduced thickness region 5008 of the example of FIG. 5E may be positioned within approximately 1 mm to approximately 5 mm from the edge 5006, whereas in the example of FIG. 5D the reduced thickness region 5008 may be positioned within approximately 1 mm to approximately 15 mm from the edge 5006.

The reduced thickness region 5008, can be positioned in any suitable surface of the seal-forming structure 3100. In previous examples the reduced thickness region 5008 has been provided on an underside (e.g. non patient contacting side 5010) of the patient contacting surface 3100A, or in the support layer 3100B. In contrast FIG. 5F provides one example of the technology in which the reduced thickness region 5008, is provided in the patient contacting surface of the seal-forming structure 3100.

### 5.3.13.1 Reduced Thickness Profiles

FIG. 6A shows the cross-sectional profile of a seal-forming structure 3100 of a patient interface. FIGS. 6B to 6E show various cross-sectional profiles of a seal-forming structure 3100 comprising a reduced thickness region 5008 in accordance with the present technology. For example, the cross sections of FIGS. 6B to 6E may substantially correspond to the region marked “A” in FIG. 5B.

In the example of FIG. 6B the reduced thickness region 5008 is provided in the form of a channel or groove having a curved, arcuate or otherwise substantially semi-circular cross-sectional shape. In some examples of the technology, this semi-circular cross-sectional shape may have a radius of curvature of between approximately 1 mm and approximately 0.25 mm, such as between approximately 0.65 mm and 0.45 mm or approximately 0.55 mm.

FIG. 6C shows a reduced thickness region 5008 having a substantially triangular cross-sectional shape. The triangular cross-sectional shape need not be a symmetrical, and could include for example saw-toothed profiles. FIG. 6D shows a reduced thickness region 5008, having a substantially trapezoidal cross-sectional shape, and FIG. 6E shows a reduced thickness region 5008, having a substantially rectangular cross sectional shape.

In each example the reduced thickness region 5008 has a thickness T1, which is less than the thickness of the adjacent sections T2 and T3, i.e. the sections of the seal-forming structure between the edge 5006 and reduced thickness region 5008 (T2), as well as the thickness between the side-wall 5002 and reduced thickness region 5008 (T3).

It should be appreciated that the thicknesses T1, T2 and T3 may vary around the perimeter of the seal-forming structure 3100. For example, in the cheek or side-of-nose regions the thickness T2 may be approximately 1 mm while the thickness T1 may be 0.5 mm. In contrast in the nasal bridge, upper lip and/or chin regions of the seal-forming structure the thickness T2 may be approximately 0.5 mm and the thickness T1 may be substantially 0.25 mm. In examples of the technology, the thickness T3 increases as it approaches, and transitions to the side-wall 5002. In some examples of the technology the thickness T3 is substantially equal to, or slightly greater than the thickness T2 when measured adjacent to the reduced thickness region 5008.

In some examples of the technology the reduced thickness region 5008 may vary by up to 1 mm in depth (T1), for example the reduced thickness region 5008 may be thinner in areas such as the nasal bridge region of the seal-forming structure 3100, and thicker in the side of nose regions of the seal-forming structure 3100. For example, the reduced thickness region 5008 may be between approximately 0.5 mm and approximately 0.2 mm in the nasal bridge region, and between approximately 1 mm and approximately 0.5 mm in the side of nose regions of the seal-forming structure 3100.

In some examples of the technology, the depth or thickness T1 of reduced thickness region 5008 in the chin, or lower lip regions may be greater than the depth or thickness of reduced thickness region 5008 in any one or more of the cheek regions, upper lip regions, alar regions, side of nose regions, nasal bridge or pronasale region of the seal-forming structure 3100. Providing a greater depth in the chin region of the seal-forming structure 3100 may advantageously encourage the flow of material in this region during manufacture, and/or reduce the flexibility of the seal-forming structure 3100 in this region.

In some examples of the technology, the depth or thickness T1 of reduced thickness region 5008 in the alar regions of the seal-forming structure 3100, may be greater than the depth or thickness of reduced thickness region 5008 in any one or more of the cheek regions, upper lip regions, chin or lower lip region, side of nose regions, nasal bridge or pronasale region of the seal-forming structure 3100. Providing a greater depth in the alar regions of the seal-forming structure 3100 may advantageously encourage the flow of material in this region during manufacture, and/or reduce the flexibility of the seal-forming structure 3100 in this region.

In each of the foregoing examples the reduced thickness region 5008 of the seal-forming structure 3100 is provided on an inner surface, i.e. the non-patient contacting side 5010, or plenum chamber side of the seal-forming structure, such that the change in thickness is less apparent to the patient 1000, than if the reduced thickness region 5008 was provided on the patient contacting side 5013 of the seal-forming structure 3100.

In examples, the width of the reduced thickness region 5008 may have a thickness of approximately 1 mm or less, for example approximately 0.5 mm or less, such as approximately 0.1 mm. In some examples, the reduced thickness region 5008 may be at least 20 mm long, more preferably at least 40 mm long. In examples, the ratio of the length of the reduced thickness region 5008 to the width of the reduced thickness region 5008 may be at least 20:1, for example, at least 50:1, for example at least 100:1.

The ratio of thickness of the reduced thickness region 5008 to thickness of the immediately adjacent portions may be 2:3 or less, for example 1:2 or less.

### 5.3.13.2 Composite Seal-Forming Structures

In some forms of the technology, the seal-forming structure 3100 comprises a plurality of layers, for example a first layer of patient contacting material and a second, non patient-contacting support layer. For example, in FIG. 5B the support layer 3100B and patient contacting layer 3100A are both formed from a resilient material such as silicone.

In other forms of the technology such as those shown in FIG. 7A, the seal-forming structure 3100 may comprise a first layer 3100A configured to engage and seal with the face of the patient 1000 in use, and a second layer 3100B configured to provide support to at least a portion of the first layer 3100A in use, wherein the first layer 3100A is attached to the second layer 3100B. In the example of FIG. 7A, the first layer 3100A is attached to the second layer 3100B along a portion of its length in the radial direction, i.e. a direction extending radially outwardly of the opening 5004. For example, the first layer 3100A may be attached to the second layer such that at least 10% of the first layer is attached to the second layer 3100B, for example in some forms approximately 50% of the first layer may be attached to the second layer. In examples, the first and second layers 3100A, 3100B may form a laminate.

In certain forms of the technology, it may be advantageous to combine the composite, multi-layered seal-forming structure 3100 with the reduced thickness regions 5008 described herein. For example, the first layer 3100A may be attached to the second layer 3100B in an area proximal to the reduced thickness region 5008.

For example, with reference to FIG. 7A the seal-forming structure 3100 may comprise a first portion/layer 3100A made from a first material (such as a textile), where the first layer includes at least one opening 5004 through which the flow of breathable gas is provided to one or more of the patient's airways in use. The seal-forming structure 3100 may further comprise a second portion/layer 3100B comprising a second material (such as a silicone), wherein the second portion 3100B is joined to the first portion 3100A, the second portion having at least one groove or channel 5008 therein, wherein the groove or channel 5008 is arranged generally parallel to at least a portion of an outer perimeter of the first material (i.e. lies along a path which extends in a direction which is generally parallel).

In another example, the seal-forming structure 3100 may comprise a first layer 3100A (which may be referred to as a patient contacting portion) comprising a first material (such as a textile), wherein the first material comprises a first/patient contacting side 5013 configured to engage the patient's face to provide the seal, and a second/non-patient contacting side 5010 which is opposite to the first/patient contacting side 5013, or otherwise facing inwardly toward the plenum chamber. In some examples the seal-forming structure 3100 further comprises a second layer 3100B (which may be referred to as a support portion) attached to the non-patient contacting side 5010 of the patient contacting portion 3100A, the support portion comprising a second material (such as a silicone) which is different from the first material. In this example the support portion may be configured to support at least part of the patient contacting portion 3100A and may comprise at least one reduced thickness region 5008 which has a thickness T1 which is less than a thickness of an adjacent region (T2 and/or T3) of the support portion 3100B, the reduced thickness region 5008 lies along a path that is generally parallel to an outer perimeter of the first material.

In other words, the second layer 3100B (support portion) may comprise a first region (reduced thickness region 5008) and a second region (adjacent region T2), the first region being thinner than the second region, and further wherein the second region is closer to the edge 5006 of the seal-forming structure than the first region.

In certain forms, the first layer 3100A may be at least partially attached to the second layer 3100B by a moulding process (such as insert moulding or over-moulding), by an adhesive, by ultrasonic welding, by stitching, or by any other suitable attachment mechanism such as hook and loop fasteners.

In one form of the technology the first layer 3100A comprises a textile material, while the second layer comprises a flexible material (e.g., constructed from a soft, flexible, resilient material like silicone, or foam, etc.).

In some forms of the technology the first layer 3100A may include a first region which is joined to the second layer 7002B, and a second unsupported region 7002 which is substantially unsupported or otherwise not joined to the second layer 3100B. The use of an unsupported region 7002 may assist in preventing the second layer 3100B from contacting the patient's face in use. For example, for some patient's use of textile materials may be more comfortable, or otherwise result in less allergenic issues than using silicone materials.

In some examples of the technology, such as those shown in FIG. 7B, the reduced thickness region 5008 may be provided with an arcuate profile having radius of curvature of between approximately 1 mm and approximately 0.25 mm, such as between approximately 0.65 mm and 0.45 mm or approximately 0.55 mm, the arc extending through an angle of between approximately 45 degrees and approximately 75 degrees such as substantially 55 degrees.

In some examples, the thickness of the second layer 3100B adjacent to the unsupported first layer 3100A (T2) may be greater than the thickness of the second layer 3100B on the opposing side (T3) of the reduced thickness region 5008. In other words the second layer of material may have a first thickness (T2) adjacent to the opening 5004, such as within 5 mm of the opening 5004, a second thickness (T1) located at a greater distance from the opening 5004 than the first thickness, such as within 10 mm from the opening 5004, and a third thickness (T3) located at a greater distance from the opening 5004 than the second thickness, such as within 15 mm of the opening 5004.

In other words, the second layer may comprise a first thickness and a second thickness, wherein the first thickness is adjacent to the second region (unsupported region) of the first layer, and the second thickness is both adjacent to the first thickness, and further from the second region than the first thickness, wherein first thickness is greater than the second thickness.

In some examples, the increased thickness of the second layer 3100B adjacent to the opening 5004 may advantageously provide for reduced resistance to material flow in an injection moulding tool, for example, where the injection point is located near to the opening 5004, as shown in FIG. 9A, this region may allow for material to flow around a perimeter of the opening 5004, before flowing past the reduced thickness region 5008.

### 5.3.13.3 Composite Seal-Forming Structure Manufacture

FIGS. 8A and 8B show one example of a composite patient interface 3000 comprising a first layer 3100A comprising a textile, which is attached to a second layer 3100B comprising silicone. In this example, seal-forming structure 3100 has a reduced thickness region 5008 which has an elongate profile and extends in a circumferential direction around the seal-forming structure in the side of nose regions, and nasal bridge region. In this example the reduced thickness region 5008 is not provided in the upper lip region of the seal-forming structure 3100.

In the example of FIGS. 8A and 8B, the first layer 3100A is attached to the second layer 3100B by an injection moulding process. For example, the first (textile) layer may be positioned within a cavity in a moulding tool, and the second layer injection moulded around the textile layer. This process should be familiar to those skilled in the art and may be known as insert or over-moulding.

One difficulty of moulding different materials using insert or over moulding processes is that the layer being insert or over moulded can move within the tool. This can be particularly problematic where the material being insert moulded is flexible, such as when using fabric or textile materials. For example, where fabric or textile materials are used, the forces imparted to the fabric or textile due to the flow of thermoset/thermoplastic material (such as silicone) within the moulding tool can cause the textile to move or dislodge from its intended location.

According to one form of the technology, a cushion module 3150 is provided which comprises a first layer 3100A such as a first textile layer and a second layer 3100B, such as a silicone layer, the textile layer being attached to the silicone layer during a moulding process. In examples, the silicone layer is attached on a first side 5013 to the textile layer, and has a reduced thickness region 5008 on a second/non-patient contacting side 5010, opposite to the textile layer.

The reduced thickness region 5008 may advantageously cause flow restriction within the injection mould tooling, to thereby direct the flow of the thermoset/thermoplastic material in order to minimise the forces which would otherwise act on the textile to displace the textile within the tooling.

FIGS. 9A to 9C show various forms of the technology in which the cushion module 3150 can be moulded to include the reduced thickness region 5008. For sake of clarity the first layer 3100A has not been shown in these figures. In each of these examples, a sprue 9002 is shown by way of example only. It should be appreciated that the sprue 9002 is removed after manufacture, and acts as a channel to provide the material to be moulded within an injection moulding tool.

In the illustrated examples, the sprue 9002 is provided in the patient contacting surface 3100 between the opening 5004 and the reduced thickness region 5008. In this way the injection moulded material is configured to flow through the patient contacting portion of the seal-forming structure 3100, radially outwardly of the opening 5004. As the sprue 9002 is provided to both sides of the seal-forming structure 3100 the forces imparted by the flowing injection moulding material should largely be equal and opposite to one another to thereby prevent distortion or displacement of the textile layer 3100A (which is not shown for sake of clarity, but in use would be at least partially attached to the second layer 3100B).

The reduced thickness region 5008 may be formed by any suitable geometry within the tooling, and can act as a flow restrictor or dam encouraging the injected material to travel around the perimeter of the seal-forming structure, rather than continuing to flow radially outwardly. This results in a continuous connection between the first layer 3100A and second layer 3100B, preferably before other areas of the seal-forming structure 3100 are moulded. One potential advantage of this approach is that it encourages the injection moulded material to fill the thin patient contacting region of the seal-forming structure 3100 first, securing the first layer 3100A to the second layer 3100B. This can help prevent a situation where the injection moulded material fills the thicker components of the seal-forming structure 3100 first, and backfills into the thinner regions of the seal-forming structure which can result in displacement of the first layer 3100A.

Another potential advantage of the reduced thickness region 5008 is that it can provide an increased pressure, and therefore force, which holds the textile against the cavity of the tool in which it has been inserted.

The amount of flow restriction provided by the reduced thickness region 5008 is a function of the width and depth of the reduced thickness region 5008. For example, in the example of FIG. 9A the channel has a substantially consistent width of approximately 0.1 mm to 0.2 mm. In FIG. 9B the reduced thickness region 5008 is wider in the side of nose regions (for nasal patient interfaces) or cheek regions (in the case of full-face patient interfaces), for example 0.2 mm to 0.5 mm. By adjusting the depth and thickness of the reduced thickness region 5008 the flow of material within the tooling can be controlled so as to reduce potential bunching or displacement of the first layer 3100A material. Similarly the depth of the reduced thickness region 5008 may be adjusted to achieve desirable flow characteristics. For example the depth of the reduced thickness region 5008 may be between 0.1 and 0.3 mm.

The reduced thickness regions 5008 may further be used to influence the end-of-fill position within the moulded part to control the location of weld lines, and to improve features such as overflow locations.

The person skilled in the art should be familiar with simulation software which allows for flow simulations to be performed, such as Moldflow® by Autodesk®.

### 5.3.14 Composite Cushion Module Structure

FIGS. 10A-10F show a cushion module 3150 according to one example of the present technology. The cushion module 3150 comprises a chassis portion 3210 and a seal-forming structure 3100 in this example. The chassis portion 3210 and seal-forming structure 3100 form a cushion module 3150 having certain features according to the present technology described herein. It is to be understood that such features may be applied to a cushion of a patient interface 3000 whether the cushion is attached to a chassis portion 3210 or not. In some examples, a seal-forming structure 3100 may form an entirety of a cushion, for example if the seal-forming structure 3100 is attached to and supported by a shell. Accordingly, it is to be understood that features described herein as being features of a cushion may be applied to a seal-forming structure 3100 (and vice versa). Likewise, while features may be described as features of a cushion module 3150, they are to be understood to be applicable to a cushion whether it is part of a removeable/separable cushion module 3150 or not. In some examples a cushion module 3150 may not be separable from other components of a patient interface 3000.

The cushion module 3150 shown in FIGS. 10A-10F forms part of a patient interface 3000 (shown in FIG. 16B). The patient interface 3000 comprises a plenum chamber 3200 pressurisable to a therapeutic pressure of at least 6 cmH2O above ambient air pressure, the plenum chamber 3200 comprising at least one plenum chamber inlet port being sized and structured to receive a flow of air at the therapeutic pressure for breathing by a patient 1000. In this example the cushion module 3150 is hollow and defines the plenum chamber 3200 within its hollow interior. The patient interface 3000 further comprises a seal-forming structure 3100 constructed and arranged to form a seal with a region of the patient's face surrounding an entrance to the patient's airways, the seal-forming structure having at least one opening 5004 therein such that the flow of air at the therapeutic pressure is delivered to at least an entrance to the patient's nares. The seal-forming structure 3100 may be constructed and arranged to maintain said therapeutic pressure in the plenum chamber throughout the patient's respiratory cycle in use. The patient interface 3000 further comprises a vent 3400 to allow a flow of gases exhaled by the patient 1000 from an interior of the plenum chamber to ambient, said vent being sized and shaped to maintain the therapeutic pressure in the plenum chamber in use. In the example shown in FIGS. 10A-10F, the chassis portion 3210 and the seal-forming structure 3100 of the cushion module 3150 together form the plenum chamber 3200, the seal-forming structure 3100 is a portion of the cushion module 3150 and will be described in more detail below, and the vent 3400 is provided by a vent module 3410 (shown in FIG. 15) able to be fitted to the chassis portion 3210 in the anterior hole 3215.

The chassis portion 3210 in this example comprises a pair of laterally projecting connection portions 3212 each defining a respective plenum chamber inlet port and each being configured to connect to a respective one of a pair of gas delivery tubes 3350. The plenum chamber inlet ports 3212 are openings at the end of the laterally projecting connection portions 3212. Furthermore, in this example, the cushion module 3150 comprises connectors 3214 to releasably fluidly connect to the gas delivery tubes 3350. In the example shown in FIG. 16B, the patient interface 3000 comprises a pair of gas delivery tubes 3350 forming part of a positioning and stabilising structure 3300 configured to provide a force to hold the seal-forming structure 3100 in a therapeutically effective position on the patient's head, each gas delivery tube 3350 being configured to convey the flow of air from a location atop the patient's head to the plenum chamber 3200 in use. As shown in FIG. 16B, the patient interface 3000 comprises a connection port 3600 atop the patient's head to connect to an air circuit 4170 connected to an RPT device 4000.

In other examples, the patient interface 3000 comprises a positioning and stabilising structure 3300 that does not comprise gas delivery tubes 3350 and instead comprises headgear straps which hold the seal-forming structure 3100 in a sealing position on the patient's face. In such forms the laterally projecting connection portions 3212 may receive a flow of air or breathable gas from tubes extending inferiorly from the patient interface 3000 or extending superiorly and attached to the headgear straps of the positioning and stabilising structure 3300. Alternatively, the cushion module 3150 may not have laterally projecting connection portions 3212 and the patient interface 3000 may instead comprise a connection port 3600 at an anterior location, such as at the location of the vent module 3410. In such a configuration the connection port 3600 and vent 3400 may be provided by the same component. FIG. 16D shows various possible configurations of a patient interface 3000 in which aspects of the present technology may be applied.

By way of example only, the patient interface 3000 and in particular the cushion module 3150, chassis portion 3210, laterally projecting connection portions 3212, connectors 3214, vent module 3410, positioning and stabilising structure 3300 may comprise any one or more features described in WO 2021/012005 (the entire contents of which are hereby incorporated herein by reference).

In the examples shown in FIGS. 10A-10F and 17A-17E, the seal-forming structure 3100 comprises a first layer 3100A on a posterior side of the seal-forming structure 3100, the first layer 3100A configured to seal in use around the entrance to the patient's airways including against an at least partially inferior-facing portion of the patient's pronasale, against the nasal alae and against the lip superior of the patient's face in use. In the example shown in FIGS. 10A-10F, the first layer 3100A seals against the lip superior from one lateral side across the mid-sagittal plane to the other lateral side. FIGS. 17A-17E show a similar example. In other examples the first layer 3100A may seal against the lip superior by sealing only to lateral portions of the lip superior. The seal-forming structure 3100 in the examples shown in FIGS. 10A-10F and 17A-17E further comprises a second layer 3100B connected to the first layer 3100A. In these examples the first layer 3100A is formed from a textile material. The second layer 3100B in these examples is formed from an elastomeric material. In examples the elastomeric material may be silicone or may be a thermoplastic elastomer (TPE). The seal-forming structure 3100 may take any form disclosed herein and may be formed from any material or combination of materials disclosed herein.

### 5.3.14.1 Lap Joint

The second layer 3100B of the seal-forming structure 3100 may comprise an overlapping portion 3102 overlapping with the first layer 3100A of the seal-forming structure to form a lap joint with the first layer 3100A around a periphery of the first layer 3100A, such as an entire periphery of the first layer 3100A, a majority of the periphery of the first layer 3100A or at least some of the periphery of the first layer 3100A.

The lap joint may be in the manner of overlap between the first layer 3100A and second layer 3100B shown in FIG. 7 or 8B, for example. FIGS. 11A and 11B show the cushion module 3150 with the first layer 3100A removed in order to show features of the second layer 3100B, in particular the overlapping portion 3102 of the second layer 3100B. As shown in FIGS. 11A and 11B, the overlapping portion 3102 of the second layer 3100B of the seal-forming structure extends inwardly, with respect to the periphery of the first layer 3100A, from a non-overlapping portion 3104 of the second layer 3100B. For clarity in FIGS. 11A and 11B the overlapping portion 3102 is lightly hatched and the non-overlapping portion 3104 is hatched more darkly. The overlapping portion 3102 of the second layer 3100B may alternatively be described as extending inwardly with respect to the outer periphery of the seal-forming structure 3100 or extending inwardly towards the centre of the cushion module 3150 or towards the one or more holes provided in the first layer 3100A to provide the flow air to the patient's airways.

### 5.3.14.1.1 Width of Lap Joint

With reference to FIGS. 11A-11B and 13A-13D, the overlapping portion 3102 may comprise a width W which varies along the overlapping portion 3102 around the periphery of the first layer 3100A. The width W may be defined by the amount of inward extension of the overlapping portion 3102 from the non-overlapping portion 3104 of the second layer 3100B. The width W is therefore also the dimension of the overlapping portion 3102 in the transverse direction with respect to the length of the overlapping portion 3102 along the path around the periphery of the first layer 3100A or around an inner periphery of the non-overlapping portion 3104. The width W of the overlapping portion 3102 at a position on the overlapping portion 3102 may also be described as the distance between the inner edge of the overlapping portion 3102 and the outer edge of the overlapping portion 3102 coinciding with the inner edge of the non-overlapping portion 3104. The width W is labelled in FIGS. 13B and 13D.

As shown in FIGS. 11A-11B, 13A-13D and 17D in particular, the overlapping portion 3102 may comprise a pair of lateral nasal portions 3106. The width W of the overlapping portion 3102 may be larger in the lateral nasal portions 3106 than in one or more other portions of the overlapping portions 3102. A greater width W in the lateral nasal portions 3106 may advantageously provide for a robust connection between the first layer 3100A and the second layer 3100B. The width W of the overlapping portion 3102 may be larger in the lateral nasal portions 3106 than in a superior portion 3107 of the overlapping portion 3102. For example, as shown by comparing FIGS. 13B and 13D, the width W of the lateral nasal portion 3106 shown in FIG. 13B is larger than the width W of the superior portion 3107 shown in FIG. 13D. The greater width W in the lateral nasal portions 3106 also provides additional stiffness to the seal-forming structure 3100 in comparison to the stiffness of the first layer 3100A alone, which may advantageously prevent creases from forming or from propagating laterally along the seal-forming structure 3100 past the patient's nasal alae in use.

A lesser width W in the superior portion 3107 of the overlapping portion 3102 may advantageously reduce the overall stiffness of the seal-forming structure 3100 in the region which seals to inferior and/or partially anterior surfaces of the patient's pronasale. This region on the patient's face may be somewhat sensitive and the lesser width of the overlapping portion 3102 may keep the pressure on this region of the patient's face low, providing for patient comfort in use. In some examples the overlapping portion 3102 may be at least twice as wide in the lateral nasal portions 3106 than in the superior portion 3107. In some examples the overlapping portion 3102 may be 1¼, 1½, 1¾, 2, 2¼, 2½, 2¾ or 3 or more times as wide in the lateral nasal portions 3106 than in the superior portion 3107.

The width W of the overlapping portion 3102 may be larger in the lateral nasal portions 3106 than in an inferior portion 3108 of the overlapping portion 3102. A lesser width W in the inferior portion 3108 of the overlapping portion 3102 may advantageously reduce the overall stiffness of the seal-forming structure 3100 in the region which seals to the patient's lip superior (the lip superior portion 3116). This region on the patient's face may be somewhat sensitive and the lesser width W of the overlapping portion 3102 may keep the pressure on this region of the patient's face low, providing for patient comfort in use. In some examples the overlapping portion 3102 may be at least five times or at least 10 times as wide in the lateral nasal portions 3106 than in the inferior portion 3107. In some examples the overlapping portion 3102 may be 4, 6, 8, 10, 12, 14, 16, 18 or 20 times as wide in the lateral nasal portions 3106 than in the inferior portion 3107. In the example illustrated in FIGS. 10A-10F and 11A-11B, the inferior portion 3107 of the overlapping portion 3102 is so small in comparison to the lateral nasal portions 3106 that it is not perceptible in the drawings shown in FIGS. 11A-11B. The small overlap between the first layer 3100A and the second layer 3100B provides the strength of a lap joint while keeping the overall stiffness of the seal-forming structure 3100 low. Furthermore, as shown in FIGS. 11A and 11B, the width W of the overlapping portion 3102 is larger in the superior portion 3107 than in the inferior portion 3108 of the overlapping portion 3102.

In some examples of the present technology there may be no overlapping portion 3102 in a lip superior portion of the seal-forming structure 3100 and the connection between the first layer 3100A and the second layer 3100B may be a butt joint between the two layers. This may advantageously even further reduce pressure on the patient's lip superior in use.

In some examples of the present technology, the overlapping portion 3102 also comprises a pair of mid-lateral inferior portions 3109 located on respective lateral sides of the inferior portion 3108 of the overlapping portion 3102 and located medially of the lateral nasal portions 3106 of the overlapping portions 3102. The width W of the overlapping portion 3102 may be larger in the lateral nasal portions 3106 than in the mid-lateral inferior portions 3109. This is shown in FIGS. 11A-11B for example. In some examples, the overlapping portion 3102 may be at least 1.5 times as wide in the lateral nasal portions 3106 than in the mid-lateral inferior portions 3109. In some examples the overlapping portion 3102 may be at least 1¾ 2, 2¼, 2½, 2¾ or 3 times as wide in the lateral nasal portions 3106 than in the mid-lateral inferior portions 3109. As shown in FIGS. 11A and 11B for example, the width W of the overlapping portion 3102 is larger in the mid-lateral inferior portions 3109 than in the inferior portion 3108 of the overlapping portion 3102.

In other examples there may be no mid-lateral inferior portions 3109 and the lateral nasal portions 3106 may be immediately adjacent an inferior portion 3108 across the lip superior portion 3116 of the seal-forming structure 3100, or there may be no mid-lateral inferior portions 3109 or inferior portions 3108.

In the example shown in FIGS. 17A-17E, the seal-forming structure 3100 comprises a nasal portion configured to provide the flow of air to the entrance to the patient's nares in use. In this example, the overlapping portion 3102 comprises a pair of lateral lip superior portions 3124 provided at respective lateral sides of the patient's lip superior in use. As shown in FIG. 17D in particular, the width of the overlapping portion 3102 is larger in the lateral lip superior portions 3124 than in the lateral nasal portions 3106. The width of the overlapping portion 3102 may be generous in the lateral lip superior portions 3124 to provide good support and/or provide for a robust seal proximate the inferior corners/alar crest points of the patient's nose, which may be regions prone to leaks. For example, the overlapping portion 3102 may be at least 1.5 times as wide in the lateral lip superior portions 3124 than in the lateral nasal portions 3106. As shown in FIG. 17D, the lateral lip superior portions 3124 extend medially of the periphery of the first layer 3100A from a non-overlapping portion 3104 of the second layer 3100B. Each lateral lip superior portion 3124 comprises a medial end positioned proximate and inferior to a respective one of the alar crest points on the patient's face. This may advantageously leave the portion of the first layer 3100A that seals to the medial portion of the patient's lip superior, more flexible, which may provide for good comfort levels in use as the medial portion of the patient's lip superior may be a sensitive area. As shown in FIG. 17D for example, the width of the overlapping portion 3102 tapers between the lateral lip superior portions 3124 and the lateral nasal portions 3106.

Also with reference to FIG. 17D, in some examples the overlapping portion 3102 comprises a pair of nasolabial portions 3120 each located laterally of a respective one of the lateral lip superior portions 3124, the width of the overlapping portion 3102 being larger in the lateral lip superior portions 3124 than in the nasolabial portions 3120. Additionally, or alternatively, the width of the overlapping portion 3102 may be larger in the lateral nasal portions 3106 than in the nasolabial portions 3120.

In the example shown in FIG. 17D, the overlapping portion 3102 comprises a pair of cheek portions 3130, the width of the overlapping portion 3102 being larger in the lateral lip superior portions 3124 than in the cheek portions 3130. The overlapping portion 3102 in this example further comprises a lip inferior portion 3122, the width of the overlapping portion 3102 being larger in the lateral lip superior portions 3124 than in the lip inferior portion 3122. In examples, the width of the overlapping portion 3102 may be largest in the lateral lip superior portions 3124.

As shown in FIG. 11B and FIG. 17D in particular, the overlapping portion 3102 may get narrower (e.g. the width W may decrease) in the lateral nasal portions 3106 towards the superior portion 3107. The widths of the lateral nasal portions 3106 may taper towards the superior portion 3107 of the overlapping portion 3102. In some forms of the present technology, there are not discrete changes in width W between the different portions of the overlapping portion 3102 but instead there are gradual changes. The overlapping portion 3102 may have an inner edge which may generally follow the shape of the boundary between the overlapping portion 3102 and the non-overlapping portion 3104, although may deviate in places to provide for changes in width W of the overlapping portion 3102. The overlapping portion 3102 may comprise rounded corners between the lateral nasal portions 3106 and the superior portion 3107 and/or between the lateral nasal portions 3106 and the mid-lateral inferior portions 3109. In some examples the overlapping portion 3102 may have rounded corners between the inferior portion 3108 and the lateral nasal portions 3106 or, if present, between mid-lateral inferior portions 3109 and the inferior portion 3108.

### 5.3.14.1.2 Profile of Overlapping Portion and Non-Overlapping Portion

FIGS. 13A-13D show cross sections through the overlapping portion 3102 and non-overlapping portion 3104 of the second layer 3100B. The first layer 3100A is not shown in FIGS. 13A-13D. FIG. 13B shows the second layer 3100B at a lateral nasal portion 3106 of the overlapping portion 3102 and FIG. 13D shows the second layer 3100B in the superior portion 3107 of the overlapping portion 3102.

As shown in each of FIGS. 13B and 13D, the overlapping portion 3102 of the second layer 3100B comprises an exterior surface 3103 to which the first layer 3100A is connected, the exterior surface 3103 of the overlapping portion 3102 being offset from and shy of an exterior surface 3105 of the non-overlapping portion 3104. Alternatively stated, the exterior surface 3105 of the non-overlapping portion 3104 is offset from and proud of an exterior surface 3103 of the overlapping portion 3102. Once the first layer 3100A is attached to the second layer 3100B, a patient-facing surface of the first layer 3100A may be flush with the exterior surface 3105 of the non-overlapping portion 3104 of the second layer 3100B. The offset between the external surfaces 3103 and 3105 allows for the first layer 3100A to be flush with the external surface 3105 of the non-overlapping portion 3104 once attached. This may provide for a substantially contiguous shape of the patient-facing surface of the seal-forming structure 3100, which may advantageously avoid facial marking and/or points of uncomfortable contact in use which may occur if there was a step between surfaces. The offset between the external surfaces 3103 and 3105 may be substantially equal to the thickness of the first layer 3100A so that when the first layer 3100A is attached to the second layer 3100B the patient-facing surface 3101 of the first layer 3100A is at the same level/flush with the external surface 3105 of the non-overlapping portion 3104 of the second layer 3100B. In some examples the offset between the external surfaces 3103 and 3105 may be less than the thickness of the first layer 3100A and the first layer 3100A may be compressed at the lap joint such that after attachment the external surfaces 3103 and 3105 are substantially flush. The first layer 3100A and second layer 3100B may be attached by gluing or by a moulding operation whereby the second layer 3100B is overmoulded to the first layer 3100A, by way of examples only. In either case some compression of the first layer 3100A may occur during the attachment step.

The overlapping portion 3102 of the second layer 3100B of the seal-forming structure 3100 may have a lesser thickness than the non-overlapping portion 3104 of the second layer 3100B, in one or more locations, for example at the lateral nasal portions 3106 of the overlapping portion 3102. This is shown in FIG. 13B for example and may facilitate the offset between the external surfaces 3103 and 3105. As shown in FIG. 13B, the interior surface of the second layer 3100B is contiguous but the exterior comprises a step creating the exterior surface 3103 of the overlapping portion 3102 and the exterior surface 3105 of the non-overlapping portion 3104, and the offset between them.

At the superior portion 3107 of the overlapping portion 3102, there is a step in the exterior of the second layer 3100B and offset external surfaces 3103 and 3105 in the same manner as at the lateral nasal portions 3106. However, the superior portion 3107 of the overlapping portion 3102 is proximate a central anterior portion 3115 of the seal-forming structure 3100, which is thinner than at lateral nasal portions 3106 of the seal-forming structure 3100. The superior portion 3107 has a portion that is thinner than the non-overlapping portion 3104, being the reduced thickness region 5008, and also has portions that are thicker than the non-overlapping portion 3104, being the portions on either side of the reduced thickness region 5008 in cross section, which may advantageously help to form a strong joint between the first layer 3100A and the second layer 3100B.

### 5.3.14.2 Portions of the Seal-Forming Structure

With reference to FIGS. 10A-10F, 12A-12E, and 14A-14G, the seal-forming structure 3100 may comprise various different portions. Certain portions are described below and other portions may be as described in WO 2021/012005.

The seal-forming structure 3100 may comprise a central portion 3111. The central portion 3111 may make most or all of the contact with the patient's face and may be configured to seal in use against an at least partially inferior-facing portion of the patient's pronasale, against the nasal alae and against the lip superior of the patient's face in use. In the examples shown in FIGS. 10A-10F, 12A-12E and 14A-14G, most or all of the central portion 3111 may be formed by the first layer 3100A of the seal-forming structure 3100. The central portion 3111 is formed from a textile material in these examples although may be formed from an elastomer or other suitable material in other examples. The central portion 3111 may be in a patient-facing wall of the seal-forming structure 3100. The patient interface 3000 may further comprise a second layer 3100B connected to and supporting the first layer 3100A around a periphery of the first layer 3100A. The second layer 3100B may form the anterior-facing wall of the seal-forming structure 3100. The second layer 3100B may be formed from an elastomeric material, such as silicone or TPE. The second layer 3100B may comprise an overlapping portion 3102 forming a lap joint with the first layer 3100A, such as described above. The second layer 3100B may additionally or alternatively comprise a reduced thickness region 5008 substantially as described above.

The seal-forming structure 3100 may further comprise a central anterior portion 3115 located proximate and inferior to the patient's pronasale in use, and may be in an anterior-facing wall of the seal-forming structure 3100. FIGS. 10A, 12B, 12E and 14C show the central anterior portion 3115, which may be centred in the anterior wall of the seal-forming structure 3100 and may intersect the sagittal plane of the patient's head in use. The central anterior portion 3115 may be at least partially located within a non-patient facing wall, e,g. an anterior-facing wall facing generally away from the patient's face. The central anterior portion 3115 may have an inferior boundary coincident with a superior boundary of the chassis portion 3210. The central anterior portion 3115 may be superiorly adjacent the chassis portion. The central anterior portion 3115 may have a thickness in the region of 0.2-0.5 mm, for example 0.25-0.4 mm, such as 0.25 mm or 0.3 mm. A low thickness in the central anterior portion 3115 may avoid excessive pressure on the user's pronasale region in use, which may be a sensitive area, and therefore may advantageously provide for good comfort levels in use.

The seal-forming structure 3100 may further comprise a pair of mid-lateral anterior portions 3125 in the anterior-facing wall positioned on respective lateral sides of the central anterior portion 3115. Each of the mid-lateral anterior portions 3125 as a whole may have a greater stiffness than the central anterior portion 3115. While the central anterior portion 3115 may be formed with a thin wall thickness relative to other portions, in order to keep pressure on the pronasale low, the mid-lateral anterior portions 3125 may be stiffer than the central anterior portion 3115 in order to provide a greater amount of support to the central portion 3111, e.g. the first layer 3100A in the illustrated examples.

Each of the mid-lateral anterior portions 3125 may comprise a superior portion 3126 and an inferior portion 3127. The inferior portion 3127 may have a lesser stiffness than the superior portion 3126. The lesser stiffness in the inferior portions 3127 of the mid-lateral anterior portions 3125 in comparison to the superior portions 3126 may reduce the pressure applied to the sides of the patient's nose in use, by allowing a larger amount of deformation in the anterior wall at the inferior portions 3127. A lesser stiffness may be provided by a lesser material thickness. FIGS. 12C and 12D show cutaway views through the mid-lateral anterior portions 3125 and show that in this example, the inferior portion 3127 of each mid-lateral anterior portion 3125 has a lesser thickness than the superior portions 3126 of the mid-lateral anterior portions 3125. FIGS. 12A, 12B and 12E have the inferior portions 3127 of the mid-lateral anterior portions 3125 highlighted to show their locations, shape and size. The locations of the superior portions 3126 are also indicated. In some examples the superior portions 3126 of the mid-lateral anterior portions 3125 may have a thickness in the range of 0.7-1.3 mm, 0.8-1.2 mm or 0.85-1.1 mm, for example. In some examples the inferior portions 3127 of the mid-lateral anterior portions 3125 may have a thickness of 0.3-0.7 mm, 0.4-0.6 mm or 0.5 mm, for example.

The inferior portion 3127 of each mid-lateral anterior portion 3125 may have a greater stiffness than the central anterior portion 3115. The greater stiffness may be provided by a greater material thickness and therefore the inferior portion 3127 of each mid-lateral anterior portion 3215 may have a greater thickness than the central anterior portion 3115. It follows that the superior portion 3126 of each mid-lateral anterior portion 3125 is also thicker than the central anterior portion 3115 since the superior portions 3126 are thicker than the inferior portions 3127.

The seal-forming structure 3100 may further comprise a pair of lateral posterior regions 3141. The lateral posterior regions 3141 may be provided on respective lateral posterior sides of the seal-forming structure 3100. Each lateral posterior region 3141 may extend posteriorly from the chassis portion 3210 and may curve medially into contact with the patient's face in use. In particular, and as shown in FIGS. 10C, 10D and others, each lateral posterior region 3141 may curve medially to form a respective one of a pair of posterior corners 3131 of the seal-forming structure 3100. The posterior corners 3131 may be configured to engage the patient's face proximate a respective one of the patient's nasolabial sulci in use. The lateral posterior regions 3141 may be as thick, or thicker than the mid-lateral anterior portions 3215, for example as thick or thicker than the superior portions 3126 of the mid-lateral anterior portions 3215.

The seal-forming structure 3100 may also comprise a lip superior portion 3116, as shown in FIGS. 10D, 10F and 12B for example. The lip superior portion 3116 may comprise a posterior portion 3133 configured to seal in use against the patient's lip superior. The lip superior portion 3116 may further comprise an anterior portion 3134 adjacent the chassis portion having a stiffness greater than a stiffness of the posterior portion. The greater stiffness may be provided by a greater thickness. As shown in FIGS. 12B and 12C for example, the anterior portion 3134 of the lip superior portion 3116 is thicker than the posterior portion 3133. With this arrangement the thinner posterior portion 3133 may advantageously be free to deform to form a good seal with the patient's lip superior, while the thicker anterior portion 3134 may provide good support to hold the seal-forming structure 3100 in shape. The posterior corners 3131 and/or lateral posterior regions 3141 may be thicker than the posterior portion 3133 of the lip superior portion 3116, and may for example be as thick as the anterior portion 3134 of the lip superior portion 3116. The posterior corners 3131 and/or lateral posterior regions 3141 may be relatively stiff and/or thick to help support the cushion module 3150 on the patient's face.

FIGS. 14A-14G show a cushion module 3150 according to another example of the present technology and which may be particularly suited to patients with wide noses.

In this example and as shown in FIG. 14C, the central anterior portion comprises an inferior portion 3114 and a superior portion 3113. The inferior portion 3114 may be stiffer than the superior portion 3113. The greater stiffness may be provided by a greater material thickness, as shown in FIG. 14C. In some examples, the superior portion 3113 may have a thickness in the range of 0.2-0.4 mm or 0.2-0.3 mm, such as 0.25 mm, for example. In some examples the inferior portion 3114 may have a thickness in the range of 0.3-0.8 mm, 0.4-0.7 mm, 0.45-0.6 mm, such as 0.5 mm for example. In this arrangement the inferior portion 3114 may be thick to provide good support for the central anterior portion 3115 while the superior portion 3113 is thin to conform to the user's pronasale (or at least inferior and/or partially inferior surfaces thereof). Another feature of this example of the present technology is that the height of the inferior portion 3114 of the central anterior portion 3115 is greater at the lateral sides of the central anterior portion 3115 medially within the central anterior portion. FIG. 14F shows the inside of the anterior-facing wall of the seal-forming structure 3100 with various portions highlighted, including the inferior portion 3114 and the superior portion 3113 of the central anterior portion 3115. As shown, the boundary between the inferior portion 3114 and the superior portion 3113 curves upwards towards the lateral sides of the central anterior portion 3115. The inferior portion 3114 may occupy more of the central anterior portion 3115 at the lateral sides than medially within the central anterior portion 3115.

In the example shown in FIGS. 14A-14G, the seal-forming structure 3100 may further comprise a pair of inner mid-lateral anterior portions 3128 located on respective lateral sides of the central anterior portion 3115. FIG. 14F shows these portions highlighted with darker shading than the inferior portion 3114 of the central anterior portion and the superior portion 3113 of the central anterior portion 3115. The inner mid-lateral portions 3128 may be stiffer and/or thicker than the central anterior portion 3115. For example, the inner mid-lateral portions 3128 may be thicker than the inferior portion 3114 and/or the superior portion 3113 of the central anterior portion. In some examples the inner mid-lateral portions 3128 may have a thickness of greater than 0.5 mm, such as within the range of 0.5 mm-0.8 mm, 0.5 mm-0.7 mm, or 0.5-0.65 mm, for example. The inner mid-lateral portions 3128 being thicker than the central anterior portion 3115 may provide structural stiffness to the seal-forming structure 3100 while allowing the central anterior portion 3115 to remain thin and readily conformable to the inferior of the patient's pronasale and without excessive pressure on the inferior of the pronasale.

The seal-forming structure 3100 may further comprise a pair of outer mid-lateral anterior portions 3129 located on respective lateral sides of the inner mid-lateral portions 3128. FIG. 14G shows the location of the outer mid-lateral anterior portion 3129 on one side of the cushion module 3150, highlighted in a darker shade than the inner mid-lateral anterior portion 3128, which in this example is medially adjacent the outer mid-lateral anterior portion 3129. The outer mid-lateral anterior portion 3129 may be stiffer and/or thicker than the inner mid-lateral anterior portion 3128. In some examples, the outer mid-lateral anterior portions 3129 may comprise a thickness within the range of 0.8-1.5 mm, such as within the range of 0.9-1.4 mm or 1-1.3 mm, for example. The outer mid-lateral anterior portion 3129 being thicker than the inner mid-lateral anterior portion 3128 and/or the central anterior portion 3116 may provide additional structural stiffness to the seal-forming structure 3100 while allowing the more medial portions to be thinner and apply less pressure on the nose closer to the pronasale, which may be more sensitive than other areas of the nose.

FIGS. 14C, 14D and 14E show the thickness profiles in the central anterior portion 3115, inner mid-lateral anterior portion 3128 and outer mid-lateral anterior portion 3129, respectively. As shown, the inner mid-lateral anterior portion 3128 is thicker than the central anterior portion 3115 but thinner than the chassis portion 3210. The outer mid-lateral anterior portion 3129 is thicker than the inner mid-lateral anterior portion 3128 and has approximately the same thickness as the chassis portion 3210. In some examples the outer mid-lateral anterior portion 3129 has substantially the same thickness as the lateral posterior regions 3141, which may be laterally and posteriorly adjacent the outer mid-lateral anterior portions 3129. In other examples the lateral posterior regions 3141 may be even thicker than the outer mid-lateral anterior portions 3129.

### 5.3.14.3 Nose and Mouth Cushions

In the examples of FIGS. 17A to 17E the cushion module 3150 is a nose and mouth cushion module, i.e., configured to deliver a flow of breathable gas to the mouth and nares of the patient 1000. In the illustrated example, the cushion module 3150 comprises a seal-forming structure 3100 configured to seal around the mouth and nares of the patient 1000.

In the illustrated example the forming structure 3100 comprises a first layer 3100A formed from a textile material, and a second layer 3100B formed from an elastomeric material. In examples the elastomeric material may be silicone or may be a thermoplastic elastomer (TPE). The seal-forming structure 3100 may take any form disclosed herein and may be formed from any material or combination of materials disclosed herein.

The first layer 3100A comprises an oral opening 5004A configured to communicate the flow of breathable gas to the oral airways of the patient 1000 in use, and one or more nasal opening(s) 5004B configured to communicate the flow of breathable gas to the nasal airways of the patient 1000 in use. In the example of FIGS. 17A to 17D, two nasal openings 5004B are provided, however in other examples this may be a single nasal opening 3100B, or a plurality of smaller nasal openings.

In examples comprising two or more nasal openings 5004B, it may be advantageous for the nasal openings 5004B to be connected by a bridge 17002 between each of the nasal openings 5004B. For example, the bridge may be formed of the first layer 3100A of textile material and configured to provide additional support to the first layer 3100A in the underside of nose region.

The first layer 3100A comprises a continuous section of material which surrounds the oral opening 5004A, which is configured to in use, seal around the mouth of the patient 1000. In the illustrated example, the first layer 3100A comprises: an upper lip region, configured to seal on or above an upper lip of the patient 1000 and below the nose of the patient 1000 in use; a lower lip region, configured to seal on or below a lower lip of the patient 1000 in use; and corresponding side of mouth regions connecting the upper lip region and lower lip regions, for example the side of mouth regions may be configured to engage with the lips and/or cheeks of the patient 1000 in use.

The first layer 3100A is also configured to provide a continuous section of material which surrounds the one or more nasal openings 5004B. For example, the first layer 3100A may be configured to engage with and seal against any one or more of the upper lip region, the alar, the entrances to the nares, the columella, the maxilla, and/or the pronasale. In some examples (not shown), the first layer 3100A may also comprise one or more nasal prongs configured to enter, and optionally engage with inner walls of, the nares of the patient 1000 in use.

In the illustrated example, the first layer 3100A is provided as a single continuous section of material which surrounds both the nasal openings 5004B and the oral opening 5004A. In other examples of the technology (not shown), a single opening 5004 may be provided which provides an air passageway into both the oral and nasal passageways of the patient 1000 in use. In other words, in some examples of the technology, the first layer 3100A may be configured to not seal in an upper lip region of the patient 1000 in use.

Reference herein to the use of continuous sections of material should be understood to refer to material which is extracted from a single unitary sheet of material, and does not require or include joints to connect sections of material together. The use of one or more continuous sections of material can in some cases assist with patient 1000 comfort, and/or help in maintaining an air-tight seal against the patient's face, for example by reducing pressure points and/or air leaks caused by joints in the material.

FIG. 17D shows an example nose and mouth cushion module 3150 with the first layer 3100A removed. In this example, it can be seen that the second layer 3100B of the seal-forming structure 3100 may comprise an overlapping portion 3102 overlapping with the first layer 3100A of the seal-forming structure to form a lap joint with the first layer 3100A. In the illustrated example, this lap joint is provided around a periphery of the first layer 3100A, such as an entire periphery of the first layer 3100A, however it should be appreciated that in other examples (not shown), the lap joint may instead be provided over a majority of the periphery of the first layer 3100A, such as excluding a lower-lip or chin area, or at least some of the periphery of the first layer 3100A, such as just in the nose regions of the seal-forming structure 3100.

The overlapping portion 3102 of the second layer 3100B of the seal-forming structure extends inwardly, with respect to the periphery of the first layer 3100A, from a non-overlapping portion 3104 of the second layer 3100B. The overlapping portion 3102 of the second layer 3100B may alternatively be described as extending inwardly with respect to the outer periphery of the seal-forming structure 3100 or extending inwardly towards the centre of the cushion module 3150 or towards the one or more openings 5004 provided in the first layer 3100A to provide the flow of air to the patient's nasal airway.

As previously discussed, it may be advantageous for the width of the second layer 3100B which provides the overlapping portion 3102 to vary around the periphery of the seal-forming structure 3100. For example, as illustrated in FIG. 17D, providing a lesser width in the superior portion 3107 of the seal-forming structure 3100 may advantageously reduce the overall stiffness of the seal-forming structure 3100 in the region which seals to inferior and/or partially anterior surfaces of the patient's pronasale. This region on the patient's face may be somewhat sensitive and the lesser width of the overlapping portion 3102 may keep the pressure on this region of the patient's face low, providing for patient comfort in use. In other words, the width of the overlapping region may be larger in the lateral nasal portions 3106 of the nasal sealing region, than in a superior portion 3107 of the nasal sealing region.

In some examples the width of the second layer 3100B which provides the overlapping portion 3102 may gradually reduce from an alar region of the seal-forming structure 3100 to the region which seals to inferior and/or partially anterior surfaces of the patient's pronasale. In some examples this gradual reduction in overlapping width may provide increased patient comfort, and/or a more effective therapeutic seal.

Additionally, as will be discussed herein, providing a reduced width overlapping portion 3102 in the nasolabial portions 3120 which is measured from the oral opening 5004A to an outer edge of the first layer in a region where the upper lip region of the first layer meets a side of mouth region of the first layer, may advantageously provide additional support and rigidity to the upper lip region.

In some examples the width in the overlapping region which supports the upper lip region of the seal-forming structure 3100 may be greater than the width of the seal-forming structure 3100 in other regions such as the superior region, side of mouth regions or chin regions.

As in previous examples, a reduced thickness region 5008 may be used in one or more regions of the seal-forming structure 3100 to control the flexibility of the seal-forming structure 3100, or otherwise control how the second layer 3100B is formed as part of a moulding process. In the illustrated example, the reduced thickness region 5008 is provided in a majority of the periphery of the overlapping portion 3102 of the second layer 3100B, excluding the chin, or lower lip region of the seal-forming structure 3100. In other words, in the illustrated example the seal-forming structure 3100 has a reduced thickness region 5008 in the overlapping portion 3102 of the second layer 3100B, which supports the first layer 3100A which is in contact with the side of mouth, alar, and pronasale region of the patient's face in use.

In one example of the technology, a reduced thickness 5008 region may be provided in a second layer 3100B of a seal-forming structure 3100 in a region which contacts and supports a first layer 3100A, such as a textile layer. In other examples, a reduced thickness region 5008 may be provided in a second layer 3100B of a seal-forming structure 3100, in a region which does not contact a first layer 3100A, for example to add flexibility, and/or control the flow of material in a moulding tool, rather than to facilitate connection between the first layer 3100A, and second layer 3100B. For example, the reduced thickness region 5008 may be provided in a chin region of the seal-forming structure 3100 in a location which is not bonded to the first layer 3100A.

FIG. 17E shows a partial cross-section of the wall thickness taken through section line F-F in FIG. 17C. In this example, the second layer 3100B tapers down in thickness from a non-patient contacting portion of the seal-forming structure 3100 towards a patient-contacting portion of the seal-forming structure 3100. The second layer 3100B may taper down in thickness through the overlapping portion 3102. For example, the second layer 3100B may taper continuously through the non-overlapping portion 3104 into the overlapping portion 3102. In some examples, the second layer 3100B comprises a gradual taper, decreasing in thickness from the non-overlapping portion 3104 through the overlapping portion 3104 of the seal-forming structure 3100 to, or proximate to, an inner edge of the second layer 3100B. The overlapping portion 3102 of the second layer 3100B supports and is joined to the first layer 3100A and may comprise a reduced thickness region 5008 as described herein.

### 5.3.14.4 Textile Construction

In some examples of the technology the first layer 3100A may be formed of a textile, or otherwise comprise a textile. For example, the first layer may include a textile with an elastomeric coating or backing in order to provide a substantially air-impermeable first layer 3100A.

In some examples of the technology, the first layer 3100A may comprise a textile as part of a composite construction. For example, the first layer may comprise a textile, and a substantially non-permeable material, such as an elastomeric layer, such as a silicone. The use of a non-permeable layer may advantageously allow the first layer 3100A to be substantially air impermeable, thereby allowing the first layer to form a seal with the patient's face in use. Joining of the first layer 3100A to the supporting second layer 3100B may be achieved for example by bonding the non-permeable material of the first layer 3100A, to a corresponding non-permeable material in the second layer 3100B. For example, the non-permeable material in the first layer may be an elastomer, such as a silicone, and the second layer may also comprise silicone. As such these two elastomeric layers may bond together during manufacturing, such as by an injection moulding process.

In examples of the technology, it may be advantageous for the non-permeable material to have a thickness of less than 1 mm, such as 0.3 mm or less. Providing a thin non-permeable material may advantageously maximise the elasticity of the textile and provide a textile which is has a more natural feel for the user.

Accordingly, in one example of the technology, the first layer 3100A is provided in a pre-configured shape which is designed to be received in a corresponding aperture in a moulding tool, such as an injection moulding tool.

It should be appreciated that where the first layer 3100A is bonded to the second layer 3100B in a moulding process, compression of the first layer 3100A may occur in regions where the first layer 3100A contacts the second layer 3100B, as illustrated in FIG. 7B.

An example first layer 3100A for a nose and mouth cushion module 3150 is illustrated in FIG. 18. In this example, the first layer 3100A may be formed from a substantially planar sheet of material, such as being cut from a roll or sheet. For example, the first layer may be cut using a rotary cutter, a die cutting machine, laser, CNC, a knife, or scissors.

The geometry of seal-forming structures 3100 used in cushion modules 3150 typically requires a complex three-dimensional shape which includes a combination of concave and convex curves through one or more axes, referred to herein as saddle regions. With elastomeric seal-forming structures 3100, such as those formed from silicone or similar, the complex shapes are formed as part of the moulding process. However, where one or more layers are formed from a sheet of planar material, it may be desirable to provide a first layer 3100A which is able to conform to the desired geometry of the seal-forming structure 3100, while minimising any deformations such as wrinkles or buckling which could affect the efficacy of the seal or reduce comfort for the patient 1000.

In nose and mouth interfaces, such as those shown in FIGS. 17A to 17E, the seal-forming surface 3100 configured to engage with or around the nasal region of the patient 1000 is positioned in a superior surface of the cushion module 3150, while the seal-forming structure 3100 configured to engage with the or around the mouth of the patient 1000 is provided in a posterior region of the cushion module 3150. Accordingly, where a textile first layer 3100A is used, this region of the seal-forming structure 3100 may be prone to buckling in the regions where the transition occurs.

Accordingly, in one example of the technology, the first layer 3100A is configured to have a first width ‘W1’ in a nasal region 18002 of the seal-forming structure 3100, a second width ‘W2’ in an oral region 18004 of the seal-forming structure 3100, and a third width ‘W3’ in a nasolabial portion 3120 where the first layer 3100A transitions from the nasal region to the oral region. In the third region, the width of the first layer 3100A is less than the width in the nasal region 18002 and oral region 18004. It should be appreciated that in a nose and mouth interface, the transition between the nasal region 18002 of the seal-forming structure 3100 and the oral region 18004 of the seal-forming structure 3100 occurs, in an upper lip region, adjacent to the alar of the patient's nose in use.

It should be appreciated that width in the aforementioned context is measured in a direction which is radially outwardly of the respective oral opening 5004A or nasal opening 5004B.

In other words, the first layer 3100A of the seal-forming structure 3100 comprises a first width W1, which is measured from a nasal opening 5004B radially outwardly to an outer edge of the first layer 3100A, a second width W2 which is measured radially outwardly from the oral opening 5004A to an outer edge of the first layer, and a third width W3 which is measured from the oral opening 5004A to an outer edge of the first layer in a region where the upper lip region of the first layer meets a side of mouth region of the first layer.

In another example, the third width may be measured from an outer edge of the first layer in a region adjacent to the alar of a patient 1000 in use.

In a further example the third width may be measured from an outer edge which is superior to an oral opening 5004A, and lateral to the nose of the patient 1000 in use.

In examples the third width may be measured in a nasolabial portion 3120 which is posterior and lateral to an alar region of the seal-forming structure 3100, proximate to the patient's nasolabial sulci in use.

In examples this first width W1 may measure between approximately 7 mm and approximately 20 mm, such as between approximately 10 mm and approximately 15 mm. The second width W2 may measure between approximately 5 mm and approximately 15 mm, such as between approximately 8 mm and approximately 12 mm. The third width W3 may measure between approximately 4 mm and approximately 10 mm, such as between approximately 5 mm and approximately 8 mm, such as substantially 6 mm.

By reducing the width W3 it may be possible to decouple tension in the outside edge of the nasal section of the first layer 3100A from the outside edge of the oral section of the first layer 3100A. This enables complex sealing geometries to be provided in a single continuous first layer 3100A without joining sections of material together.

In some examples, width W1 may be substantially constant, i.e., have total variation of less than 50% around nasal openings 5004B. Similarly, width W2 may be substantially constant in a chin region, and side of mouth regions, i.e., have total variation of less than 50%, while width W3 may represent a reduction in width W1 or W2, which is greater than or equal to 50%.

Given the complex three dimensional geometry of the seal-forming structure 3100, it may be appropriate for the first layer 3100A to be measured in a substantially planar configuration, for example with the first layer 3100A removed from the cushion module 3150 and placed on a substantially flat surface.

The foregoing reduced width region may allow the first layer 3100A to curve from the oral region into the nasal region with minimal buckling or wrinkling. In addition, this reduced width region can allow the first layer to assume a concave shape in the nasal region, while maintaining a substantially wrinkle free first layer 3100A.

It should be appreciated that in order to provide a substantially smooth outer surface to the seal-forming structure, in regions where the width of the first layer 3100A is reduced (such as W3), it may be advantageous for the width of the second layer 3100B to be increased to provide a substantially smooth outer surface.

In other examples of the technology (not shown) the first layer 3100A may be cut from a sheet of planar material, such as is shown in FIG. 18, then subsequently shaped into the desired three-dimensional shape prior to being attached to the second layer 3100B of the seal-forming structure 3100. For example, the first layer may be subject to a forming technology such as thermoforming prior to being positioned within the cavity of a moulding tool.

### 5.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

### 5.6 Glossary

For the purposes of the present technology disclosure, in certain forms of the present technology, one or more of the following definitions may apply. In other forms of the present technology, alternative definitions may apply.

### 5.6.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar ˜0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.6.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.6.1.2 Mechanics

### Axes:

Neutral axis: An axis in the cross-section of a beam or plate along which there are no longitudinal stresses or strains.
Longitudinal axis: An axis extending along the length of a shape. The axis generally passes through a center of the shape.
Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.6.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

Thin structures:
a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

### Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.6.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

### 5.6.3 Anatomy

### 5.6.3.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.6.4 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.6.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.6.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down).

Negative curvature: If the curve at p turns away from the outward normal, the curvature in that direction at that point will be taken to be negative (if the imaginary small person leaves the point p they must walk downhill). Such curves are often referred to as convex.

### 5.6.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A patient interface for delivering a flow of breathable gas to the airways of a patient, the patient interface comprising:
a plenum chamber pressurisable to a therapeutic pressure of at least 6 cmH2O above ambient air pressure, the plenum chamber comprising at least one plenum chamber inlet port being sized and structured to receive a flow of air at the therapeutic pressure for breathing by a patient;
a seal-forming structure constructed and arranged to form a seal with a region of the patient's face surrounding an entrance to the patient's airways, the seal-forming structure having at least one opening therein such that the flow of air at the therapeutic pressure is delivered to at least an entrance to the patient's nares, the seal-forming structure constructed and arranged to maintain said therapeutic pressure in the plenum chamber throughout the patient's respiratory cycle in use;
wherein the seal-forming structure comprises a first layer configured to engage and seal with the face of the patient in use, and a second layer configured to provide support to at least a portion of the first layer in use,
wherein the first layer includes a first region which is joined to the second layer, and a second region which is not joined to the second layer,
wherein the second layer has a first thickness and a second thickness, wherein the first thickness is adjacent to the second region of the first layer, and the second thickness is both adjacent to the first thickness, and further from the second region than the first thickness, and
wherein the first thickness is greater than the second thickness.

2. The patient interface of claim 1, wherein the first region is joined to the second layer with a lap joint.

3. The patient interface of claim 1, wherein the second layer further has a third thickness which is adjacent to the second thickness, and further from the second region than the second thickness.

4. The patient interface of claim 3, wherein the first thickness is greater than the third thickness.

5. The patient interface of claim 1, wherein the second thicknesses is provided as a channel having an elongate structure which extends in a circumferential direction around at least a portion of the seal-forming structure.

6. The patient interface of claim 5, wherein the elongate structure forms a continuous loop around the at least one opening in the seal-forming structure.

7. The patient interface of claim 1, wherein the first and second thicknesses are in one or more of: a side-of-nose region; a nasal bridge region; an upper lip region; a cheek region; a lower lip region; a chin region; an alar region; and/or a pronasale region of the seal-forming structure.

8. The patient interface of claim 1, wherein the first layer comprises a textile and the second layer comprises an elastomeric material.

9. A patient interface for delivering a flow of breathable gas to the airways of a patient, the patient interface comprising:
a plenum chamber pressurisable to a therapeutic pressure of at least 6 cmH2O above ambient air pressure, the plenum chamber comprising at least one plenum chamber inlet port being sized and structured to receive a flow of air at the therapeutic pressure for breathing by a patient;
a seal-forming structure constructed and arranged to form a seal with a region of the patient's face surrounding an entrance to the patient's airways, the seal-forming structure constructed and arranged to maintain said therapeutic pressure in the plenum chamber throughout the patient's respiratory cycle in use;
wherein the seal-forming structure comprises an edge which defines an opening such that the flow of air at the therapeutic pressure is delivered from the plenum chamber through the opening to the patient's airways,
wherein the seal-forming structure comprises a patient contacting portion configured to engage with the patient's face to provide the seal, and a support portion attached to a non-patient contacting side of the patient contacting portion, the support portion being configured to support the patient contacting portion,
wherein the support portion comprises a first region and a second region, the first region being thinner than the second region, and further wherein the second region is closer to the edge of the seal-forming structure than the first region.

10. The patient interface of claim 9, wherein the first region has a width and a length, the length being measured in a circumferential direction around the seal-forming structure, and the width being measured across a surface of the seal-forming structure in a direction substantially perpendicular to the circumferential direction, wherein the length is substantially greater than the width.

11. The patient interface of claim 9, further comprising a third region, the third region being thicker than the first region and being located further from the edge of the seal-forming structure than the first region.

12. The patient interface of claim 11, wherein the third region extends in a circumferential direction around the seal-forming structure.

13. The patient interface of claim 9, wherein the first region is provided in one or more of a: side-of-nose region; a nasal bridge region; an upper lip region; cheek region; a lower lip region; a chin region and/or a pronasale region of the seal-forming structure.

14. The patient interface of claim 13, wherein the first region is provided in the side of nose and nasal bridge regions of the patient interface.

15. The patient interface of claim 9, wherein the support portion comprises a plurality of first regions including said first region and a plurality of second regions including said second region, each of the first regions having a thickness which is less than a corresponding one of the second regions.

16. The patient interface of claim 15, wherein the plurality of first regions are provided on opposite sides of the seal-forming structure in a substantially symmetrical arrangement.

17. The patient interface of claim 9, wherein the first region forms a continuous loop around the opening in the seal-forming structure.

18. The patient interface of claim 9, wherein a region of transition from the first region to the second region has a substantially curved profile when viewed in a cross-sectional plane extending substantially perpendicular to a longitudinal direction of the first region.

19. The patient interface of claim 9, wherein the first region is provided as a channel in the non-patient contacting side of the patient contacting portion.

20.-22. (canceled)

23. A cushion module for a patient interface, the cushion module comprising:
a plenum chamber pressurisable to a therapeutic pressure of at least 6 cmH2O above ambient air pressure, the plenum chamber comprising at least one plenum chamber inlet port being sized and structured to receive a flow of breathable gas at the therapeutic pressure for breathing by a patient, and
a seal-forming structure at least partially defining the plenum chamber and being constructed and arranged to form a seal with a region of the patient's face surrounding an entrance to the patient's airways, the seal-forming structure constructed and arranged to maintain said therapeutic pressure in the plenum chamber throughout the patient's respiratory cycle in use,
wherein the seal-forming structure comprises a first portion made from a first material, at least one opening provided in the first portion through which the flow of breathable gas is provided to one or more of the patient's airways in use, and
wherein the seal-forming structure further comprises a second portion comprising a second material, wherein the second portion is joined to the first portion, the second portion having at least one groove or channel therein, wherein the groove or channel lies along a path that is generally parallel to at least a portion of the opening.

24.-72. (canceled)
